(19) United States
(12) Patent Application Publication
(10) Pub. No.: US 2025/0051794 A1
(43) Pub. Date: Feb. 13, 2025

(54) TOBACCO PLANTS HAVING INCREASED NITROGEN EFFICIENCY AND METHODS OF USING SUCH PLANTS

(71) Applicant: Altria Client Services LLC, Richmond, VA (US)

(72) Inventors: Jesse Frederick, Richmond, VA (US); Chengalrayan Kudithipudi, Midlothian, VA (US); Dongmei Xu, Glen Allen, VA (US)

(73) Assignee: Altria Client Services LLC, Richmond, VA (US)

(21) Appl. No.: 18/928,836

(22) Filed: Oct. 28, 2024

Related U.S. Application Data
(63) Continuation of application No. 17/587,441, filed on Jan. 28, 2022, now Pat. No. 12,163,139.
Application No. 17/587,441 is a continuation of application No. 15/727,418, filed on Oct. 6, 2017, now Pat. No. 11,266,174.
(60) Provisional application No. 62/405,747, filed on Oct. 7, 2016.

(51) Int. Cl. C12N 15/82; A24B 3/12; A24B 15/20; A61K 38/00; C12N 9/88; C12N 15/10; C12N 15/66

(52) U.S. Cl. CPC C12N 15/8261; A24B 3/12; A24B 15/20; C12N 9/88; C12N 15/102; C12N 15/66; C12N 15/8243; C12N 15/825; C12N 15/8269; C12Y 401/01015; A61K 38/00; Y02A 40/146

(57) ABSTRACT

This disclosure provides a number of sequences involved in nitrogen utilization, methods of using such sequences, tobacco plants carrying modifications to such sequences, tobacco plants transgenic for such sequences, and tobacco products made from such plants.

### CROSS-REFERENCE TO RELATED APPLICATIONS

This application is a Continuation of U.S. patent application Ser. No. 17/587,441, filed Jan. 28, 2022, which is a Continuation of U.S. patent application Ser. No. 15/727,418, filed Oct. 6, 2017, now U.S. Pat. No. 11,266,174, issued on Mar. 8, 2022, which claims benefit of U.S. Provisional Application No. 62/405,747, filed on Oct. 7, 2016, the disclosure of which is incorporated by reference in its entirety.

### INCORPORATION OF SEQUENCE LISTING

A sequence listing contained in the file named “P34627US02_SL.xml” which is 73,203 bytes (measured in MS Windows®), contains 20 sequences, and was produced on Oct. 18, 2024, is filed electronically herewith and incorporated by reference in its entirety.

### TECHNICAL FIELD

This disclosure generally relates to tobacco plants.

### BACKGROUND

Burley tobacco typically requires higher amounts of nitrogen fertilizer to provide the best yields. Maryland tobacco, on the other hand, requires approximately 25% of the level of nitrogen fertilizer typically used in growing burley tobacco. Fertilizer represents a significant cost in the cultivation of tobacco. Therefore, tobacco plants that have a nitrogen efficiency similar to that of Maryland tobacco are desirable.

### SUMMARY

This disclosure provides a number of sequences involved in nitrogen utilization, methods of using such sequences, tobacco plants carrying modifications to such sequences, tobacco plants transgenic for such sequences, and tobacco products made from such plants.

In one aspect, a tobacco hybrid, variety, line, or cultivar is provided that includes plants having a mutation. As disclosed herein, such a mutation can be in one or more endogenous nucleic acids having a sequence selected from the group consisting of SEQ ID NOs: 1, 2, 5, 6, 9, 10, 13, 14, 17, and 18. In some embodiments, the plants exhibit an increase in yield relative to a plant lacking the mutation. In some embodiments, the plants exhibit a decrease in the amount and/or rate of chlorophyll loss relative to a plant lacking the mutation. In still another aspect, seed produced by such a tobacco hybrid, variety, line, or cultivar, where the seed includes the mutation in one or more endogenous nucleic acids having a sequence selected from the group consisting of SEQ ID NOs: 1, 2, 5, 6, 9, 10, 13, 14, 17, and 18.

In another aspect, a method of making a tobacco plant is provided. Such a method typically includes the steps of: inducing mutagenesis in Nicotiana tabacum cells to produce mutagenized cells; obtaining one or more plants from the mutagenized cells; and identifying at least one of the plants that comprises a mutation in one or more endogenous nucleic acids having a sequence selected from the group consisting of SEQ ID NOs: 1, 2, 5, 6, 9, 10, 13, 14, 17, and 18. Such a method further can include identifying at least one of the plants that exhibits an increase in yield relative to a plant lacking the mutation. Such a method further can include identifying at least one of the plants that exhibits a decrease in the amount and/or rate of chlorophyll loss relative to a plant lacking the mutation.

In one embodiment, mutagenesis is induced using a chemical mutagen or ionizing radiation. Representative chemical mutagens are selected from the group consisting of nitrous acid, sodium azide, acridine orange, ethidium bromide, and ethyl methane sulfonate (EMS). Representative ionizing radiation is selected from the group consisting of x-rays, gamma rays, fast neutron irradiation, and UV irradiation. In some embodiments, mutagenesis is induced using TALEN. In some embodiments, mutagenesis is induced using CRISPR/Cas9. In some embodiments, mutagenesis is induced using zinc-finger nuclease technology.

In another aspect, a method for producing a tobacco plant is provided. Such a method typically includes the steps of: crossing at least one plant of a first tobacco line with at least one plant of a second tobacco line, the plant of the first tobacco line having a mutation in one or more endogenous nucleic acids having a sequence selected from the group consisting of SEQ ID NOs: 1, 2, 5, 6, 9, 10, 13, 14, 17, and 18; and selecting for progeny tobacco plants that have the mutation. In some embodiments, such a method can further include selecting for progeny tobacco plants that exhibit an increase in yield relative to a plant lacking the mutation. In some embodiments, such a method can further include selecting for progeny tobacco plants that exhibit a decrease in the amount and/or rate of chlorophyll loss relative to a plant lacking the mutation.

In still another aspect, a tobacco product is provided that includes cured leaf from a tobacco plant having a mutation in one or more endogenous nucleic acids having a sequence selected from the group consisting of SEQ ID NOs: 1, 2, 5, 6, 9, 10, 13, 14, 17, and 18.

In yet another aspect, a method of producing a tobacco product is provided. Such a method typically includes providing cured leaf from a tobacco plant having a mutation in one or more endogenous nucleic acids having a sequence selected from the group consisting of SEQ ID NOs: 1, 2, 5, 6, 9, 10, 13, 14, 17, and 18; and manufacturing a tobacco product using the cured leaves. In one embodiment, the mutation is selected from the group consisting of a point mutation, an insertion, a deletion, and a substitution.

In one aspect, a transgenic tobacco plant is provided that includes a plant expression vector, wherein the plant expression vector comprises a nucleic acid molecule that is at least 25 nucleotides in length and has at least 91% sequence identity to a sequence selected from the group consisting of SEQ ID NOs: 1, 2, 5, 6, 9, 10, 13, 14, 17, and 18. In some embodiments, expression of the nucleic acid molecule results in plants exhibiting an increase in yield relative to a tobacco plant not expressing the nucleic acid molecule. In some embodiments, expression of the nucleic acid molecule results in plants exhibiting a decrease in the amount and/or rate of chlorophyll loss relative to a tobacco plant not expressing the nucleic acid molecule. In another aspect, seed produced by such a transgenic tobacco plant is provided, where the seed comprises the plant expression vector.

In another aspect, a transgenic tobacco plant is provided that includes a heterologous nucleic acid molecule of at least 25 nucleotides in length, wherein the nucleic acid molecule hybridizes under stringent conditions to a nucleic acid sequence selected from the group consisting of SEQ ID NOs: 1, 2, 5, 6, 9, 10, 13, 14, 17, and 18. In one embodiment, expression of the heterologous nucleic acid molecule results in plants exhibiting an increase in yield relative to a plant not expressing the nucleic acid molecule. In one embodiment, expression of the heterologous nucleic acid molecule results in plants exhibiting a decrease in the amount and/or rate of chlorophyll loss relative to a plant not expressing the nucleic acid molecule. In still another aspect, seed produced by such a transgenic tobacco plant is provided, wherein the seed comprises the heterologous nucleic acid molecule.

In still another aspect, leaf from a transgenic tobacco plant is provided that includes a vector, wherein the vector comprises a nucleic acid molecule having at least 91% sequence identity to 25 or more contiguous nucleotides of a nucleic acid sequence selected from the group consisting of SEQ ID NOs: 1, 2, 5, 6, 9, 10, 13, 14, 17, and 18. In some embodiments, expression of the nucleic acid molecule results in the leaf exhibiting an increase in yield relative to leaf from a tobacco plant not expressing the nucleic acid molecule. In some embodiments, expression of the nucleic acid molecule results in the leaf exhibiting a decrease in the amount and/or rate of chlorophyll loss relative to leaf from a tobacco plant not expressing the nucleic acid molecule.

In another aspect, a method of making a transgenic plant is provided that includes expressing a transgene in the plant, wherein the transgene encodes a double-stranded RNA molecule that inhibits expression from a nucleic acid sequence selected from the group consisting of SEQ ID NOs: 1, 2, 5, 6, 9, 10, 13, 14, 17, and 18, wherein the double-stranded RNA molecule comprises at least 25 consecutive nucleotides having 91% or greater sequence identity to a sequence selected from the group consisting of SEQ ID NOs: 1, 2, 5, 6, 9, 10, 13, 14, 17, and 18. In some embodiments, expression of the transgene results in leaf from the plant exhibiting an increase in yield relative to leaf from a plant not expressing the transgene. In some embodiments, expression of the transgene results in leaf exhibiting a decrease in the amount and/or rate of chlorophyll loss relative to leaf from a plant not expressing the nucleic acid molecule. In some embodiments, the double-stranded RNA molecule comprises a sequence selected from the group consisting of SEQ ID NOs: 4, 8, 12, 16, and 20.

In still another aspect, a method of decreasing the amount and/or rate of chlorophyll loss in a tobacco plant is provided that includes the steps of introducing a heterologous nucleic acid molecule operably linked to a promoter into tobacco cells to produce transgenic tobacco cells, wherein the heterologous nucleic acid molecule comprises at least 25 nucleotides in length and has at least 91% sequence identity to a nucleic acid sequence selected from the group consisting of SEQ ID NOs: 1, 2, 5, 6, 9, 10, 13, 14, 17, and 18; and regenerating transgenic tobacco plants from the transgenic tobacco cells, wherein the transgenic tobacco plants exhibit a decrease in the amount and/or rate of chlorophyll loss. Such a method can further include selecting at least one of the transgenic tobacco plants that exhibits an increase in yield relative to a tobacco plant not expressing the heterologous nucleic acid molecule.

In another aspect, cured tobacco leaf from a transgenic tobacco plant is provided that includes a vector, wherein the vector comprises a nucleic acid molecule having at least 91% sequence identity to 25 or more contiguous nucleotides of a nucleic acid sequence such as SEQ ID NOs: 1, 2, 5, 6, 9, 10, 13, 14, 17, and 18. In some embodiments, the nucleic acid molecule has at least 95% sequence identity to SEQ ID NOs: 1, 2, 5, 6, 9, 10, 13, 14, 17, and 18. In some embodiments, the nucleic acid molecule has at least 99% sequence identity to SEQ ID NOs: 1, 2, 5, 6, 9, 10, 13, 14, 17, and 18. In some embodiments, the nucleic acid is in sense orientation. In some embodiments, the nucleic acid is in antisense orientation.

In still another aspect, a transgenic tobacco plant is provided that includes a plant expression vector, wherein the expression vector comprises a nucleic acid molecule having at least 95% sequence identity to a sequence selected from the group consisting of SEQ ID NOs: 1, 2, 5, 6, 9, 10, 13, 14, 17, and 18, or a fragment of any of those sequences encoding a functional polypeptide. In some embodiments, expression of the nucleic acid molecule or a functional fragment thereof results in plants exhibiting an increase in yield relative to tobacco plant not expressing the nucleic acid molecule or functional fragment thereof. In one aspect, seed produced by such a transgenic tobacco plant is provided that includes the expression vector.

In yet another aspect, a transgenic tobacco plant is provided that includes a heterologous nucleic acid molecule, wherein the nucleic acid molecule hybridizes under stringent conditions to a nucleic acid sequence selected from the group consisting of SEQ ID NOs: 1, 2, 5, 6, 9, 10, 13, 14, 17, and 18, or a fragment thereof encoding a functional polypeptide. In one embodiment, expression of the heterologous nucleic acid molecule or functional fragment thereof results in plants exhibiting an increase in yield relative to a tobacco plant not expressing the nucleic acid molecule or functional fragment thereof. In one aspect, seed produced by such a transgenic tobacco plant is provided that includes the heterologous nucleic acid molecule.

In one aspect, leaf from a transgenic tobacco plant is provided that includes a vector, wherein the vector comprises a nucleic acid molecule having at least 95% sequence identity to a nucleic acid sequence selected from the group consisting of SEQ ID NOs: 1, 2, 5, 6, 9, 10, 13, 14, 17, and 18, or a fragment thereof encoding a functional polypeptide. In one embodiment, expression of the nucleic acid molecule or functional fragment thereof results in the leaf exhibiting a decrease in the amount and/or rate of chlorophyll loss relative to leaf from a tobacco plant not expressing the nucleic acid molecule or functional fragment thereof. In one embodiment, expression of the nucleic acid molecule or functional fragment thereof results in the leaf exhibiting an increase in yield relative to leaf from a tobacco plant not expressing the nucleic acid molecule or functional fragment thereof.

In another embodiment, a method of decreasing the amount and/or rate of chlorophyll loss in a tobacco plant is provided. Such a method typically includes the steps of introducing a heterologous nucleic acid molecule operably linked to a promoter into tobacco cells to produce transgenic tobacco cells, wherein the heterologous nucleic acid molecule has at least 95% sequence identity to a nucleic acid sequence selected from the group consisting of SEQ ID NOs: 1, 2, 5, 6, 9, 10, 13, 14, 17, and 18, or a fragment thereof encoding a functional polypeptide; and regenerating transgenic tobacco plants from the transgenic tobacco cells, wherein the transgenic tobacco plants exhibit a decrease in the amount and/or rate of chlorophyll loss.

Such a method can further include selecting at least one of the transgenic tobacco plants that exhibits a decrease in the amount and/or rate of chlorophyll loss relative to a tobacco plant not expressing the heterologous nucleic acid molecule or functional fragment thereof. Representative methods for introducing the heterologous nucleic acid molecule into the tobacco cells using particle bombardment, Agrobacterium-mediated transformation, microinjection, polyethylene glycol-mediated transformation, liposome-mediated DNA uptake, or electroporation.

In still another aspect, cured tobacco leaf from a transgenic tobacco plant is provided that includes a vector, wherein the vector comprises a nucleic acid molecule having at least 95% sequence identity to a nucleic acid sequence selected from the group consisting of SEQ ID NOs: 1, 2, 5, 6, 9, 10, 13, 14, 17, and 18, or a fragment thereof encoding a functional polypeptide.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a graph showing chlorophyll loss in transgenic plants.

FIG. 2 is a graph showing the growth rate in transgenic plants.

FIG. 3 is a graph showing the chlorophyll content in transgenic top, middle and bottom leaves from plants.

FIG. 4 is a graph showing the green leaf yield in transgenic plants.

FIG. 5A is a graph showing amino acid content in segregating plants from a cross between TN90 and Maryland 609 grown under low nitrogen conditions (45 lb nitrogen/acre land).

FIG. 5B is a graph showing the arginine content in plants relative to chlorophyll content.

FIG. 6 is a graphical representation showing a representation of SNPs in several Burley tobacco varieties, a Maryland (MD) variety and the segregating F2 population. L, low yield; H, high yield; Y, yellow color; G, green color.

FIG. 7 is a graph showing the levels of glutamate and GABA in the roots of two GDC RNAi lines.

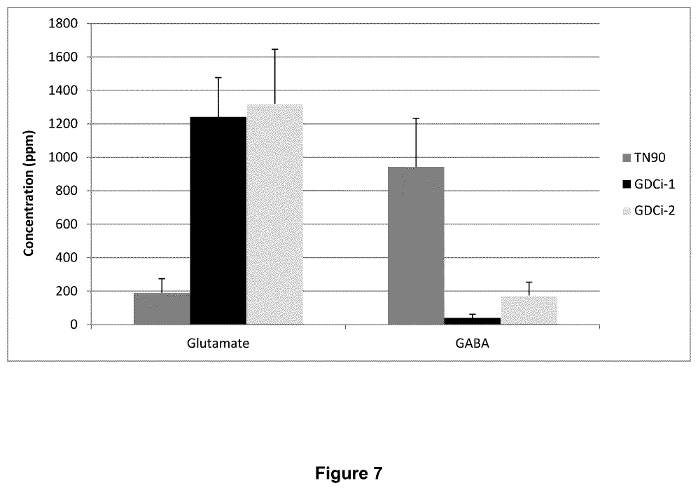

### DETAILED DESCRIPTION

This disclosure provides nucleic acids from N. tabacum that encode polypeptides associated with nitrogen use. Such nucleic acids, SEQ ID NOs: 1, 2, 5, 6, 9, 10, 13, 14, 17, and 18, and the polypeptides encoded thereby, SEQ ID NOs: 3, 7, 11, 15, and 19, are described and characterized herein. As described herein, the level of expression of such nucleic acid sequences and/or the function of such polypeptides can be modulated in N. tabacum and the resulting effect on nitrogen utilization in plants can be evaluated. Modulating polypeptide function and/or genes expression can result in an increase in yield and a decrease in the amount and/or rate of chlorophyll loss in tobacco and resulting tobacco products.

### Nucleic Acids and Polypeptides

Nucleic acids are provided herein (see, for example, SEQ ID NOs: 1, 2, 5, 6, 9, 10, 13, 14, 17, and 18). As used herein, nucleic acids can include DNA and RNA, and includes nucleic acids that contain one or more nucleotide analogs or backbone modifications. A nucleic acid can be single stranded or double stranded, which usually depends upon its intended use. The nucleic acids provided herein encode polypeptides (see, for example, SEQ ID NOs: 3, 7, 11, 15, and 19).

Also provided are nucleic acids and polypeptides that differ from SEQ ID NOs: 1, 2, 5, 6, 9, 10, 13, 14, 17, and 18 and SEQ ID NOs: 3, 7, 11, 15, and 19. Nucleic acids and polypeptides that differ in sequence from SEQ ID NOs: 1, 2, 5, 6, 9, 10, 13, 14, 17, and 18 and SEQ ID NOs: 3, 7, 11, 15, and 19, can have at least 50% sequence identity (e.g., at least 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity) to SEQ ID NOs: 1, 2, 5, 6, 9, 10, 13, 14, 17, and 18 and SEQ ID NOs: 3, 7, 11, 15, and 19.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the computer program ClustalW and default parameters, which allows alignments of nucleic acid or polypeptide sequences to be carried out across their entire length (global alignment). Chenna et al., 2003, Nucleic Acids Res., 31(13):3497-500. ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the default parameters can be used (i.e., word size: 2; window size: 4; scoring method: percentage; number of top diagonals: 4; and gap penalty: 5); for an alignment of multiple nucleic acid sequences, the following parameters can be used: gap opening penalty: 10.0; gap extension penalty: 5.0; and weight transitions: yes. For fast pairwise alignment of polypeptide sequences, the following parameters can be used: word size: 1; window size: 5; scoring method: percentage; number of top diagonals: 5; and gap penalty: 3. For multiple alignment of polypeptide sequences, the following parameters can be used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; and residue-specific gap penalties: on. ClustalW can be run, for example, at the Baylor College of Medicine Search Launcher website or at the European Bioinformatics Institute website on the World Wide Web.

Changes can be introduced into a nucleic acid molecule (e.g., SEQ ID NOs: 1, 2, 5, 6, 9, 10, 13, 14, 17, and 18), thereby leading to changes in the amino acid sequence of the encoded polypeptide (e.g., SEQ ID NOs: 3, 7, 11, 15, and 19). For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

As used herein, a “purified” polypeptide is a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the polypeptides and naturally occurring molecules with which it is naturally associated. Since a polypeptide that is chemically synthesized is, by nature, separated from the components that naturally accompany it, a synthetic polypeptide is “purified.”

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

A vector containing a nucleic acid (e.g., a nucleic acid that encodes a polypeptide) also is provided. Vectors, including expression vectors, are commercially available or can be produced by recombinant DNA techniques routine in the art. A vector containing a nucleic acid can have expression elements operably linked to such a nucleic acid, and further can include sequences such as those encoding a selectable marker (e.g., an antibiotic resistance gene). A vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6xHis tag, glutathione S-transferase (GST))

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins. As used herein, operably linked means that a promoter or other expression element(s) are positioned in a vector relative to a nucleic acid in such a way as to direct or regulate expression of the nucleic acid (e.g., in-frame). Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Vectors as described herein can be introduced into a host cell. As used herein, “host cell” refers to the particular cell into which the nucleic acid is introduced and also includes the progeny of such a cell that carry the vector. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, NY; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid.

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are pre-hybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed. Simply by way of example, high stringency conditions typically include a wash of the membranes in 0.2X SSC at 65° C.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, CA).

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

Certain of the nucleic acids described herein (e.g., SEQ ID NO:1 and 2) are predicted to encode a polypeptide (e.g., SEQ ID NO:3) that belong to the “DNA-binding One Zinc Finger” (Dof) family of sequences. “Dof zinc finger” proteins are a particular class of zinc finger domain transcription factors (TFs) characterized by a conserved region of 50 amino acids with a C2-C2 finger structure, which binds specifically to 5′-T/AAAAG-3′ DNA sequences. Dof proteins have been reported to participate in the regulation of gene expression in processes such as seed germination and seed storage protein synthesis in developing endosperm, light regulation of genes involved in carbohydrate metabolism, plant defense mechanisms, and auxin response.

Certain of the nucleic acids described herein (e.g., SEQ ID NOs:5 and 6) are predicted to encode a polypeptide (e.g., SEQ ID NO:7) that belong to the “protein TIC 22-like” family of sequences. The “protein TIC 22-like” family of polypeptides is involved in protein precursor import into chloroplasts. See, for example, UniProt F4J469 and Q9ZST9. See also, for example, Kouranov et al. (1998, J. Cell Biol., 143:991-1002) and Kouranov & Schnell (1997, J. Cell Biol., 139:1677-85).

Certain of the nucleic acids described herein (e.g., SEQ ID NOs:9 and 10) are predicted to encode a polypeptide (e.g., SEQ ID NO:11) that belong to the glutamate decarboxylase family of sequences. Glutamate decarboxylase (EC 4.1.1.15) catalyzes the decarboxylation of glutamate to GABA and carbon dioxide, using PLP as a co-factor. See, for example, UniProt Q42521 and Q7XJB3. See also, for example, Gut et al. (2009, J. Mol. Biol., 392:334-51) and Matsuyama et al. (2009, J. Biosci. Bioeng., 107:538-43).

Certain of the nucleic acids described herein (e.g., SEQ ID NOs:13 and 14) are predicted to encode a polypeptide (e.g., SEQ ID NO:15) that belong to the “mads-box TF 27-like” family of sequences. Mads-box transcription factors typically contain a highly conserved 58 amino acid long DNA binding MADS domain, and have been shown to be expressed in vegetative tissues, ovule, embryo, root and fruit, suggesting a diverse role in plant development. See, for example, XM_008671095 and XM_008671096. See also, for example, Kumar et al. (2016, Sci. Reports, 6:20695).

Certain of the nucleic acids described herein (e.g., SEQ ID NOs:17 and 18) are predicted to encode a polypeptide (e.g., SEQ ID NO:19) that belong to the PKHD-type hydroxylase family of sequences. The PKHD-type hydroxylase family (EC 1.14.11.-) typically possesses dioxygenase activity and binds iron ions and L-ascorbic acid. See, for example, UniProt Q3ED68.

### Plants Having Increased Yield and Decreased Yellowing and Methods of Making

Tobacco hybrids, varieties, lines, or cultivars are provided that have a mutation in one or more endogenous nucleic acids described herein (e.g., SEQ ID NOs: 1, 2, 5, 6, 9, 10, 13, 14, 17, and 18). As described herein, leaf from plants having a mutation in one or more of the endogenous nucleic acids (e.g., SEQ ID NOs: 1, 2, 5, 6, 9, 10, 13, 14, 17, or 18) can exhibit an increase in yield or a decrease in the amount and/or rate of chlorophyll loss (e.g., compared to a plant that lacks the mutation). In addition, plants having a mutation in one or more of the endogenous nucleic acids (e.g., SEQ ID NOs: 1, 2, 5, 6, 9, 10, 13, 14, 17, or 18) can exhibit an increase in yield or a decrease in the amount and/or rate of chlorophyll loss (e.g., compared to a plant lacking the mutation).

It would be understood by a skilled artisan that a mutation in a nucleic acid (e.g., SEQ ID NOs: 1, 2, 5, 6, 9, 10, 13, 14, 17, or 18) may or may not be reflected in the encoded polypeptide sequence (e.g., SEQ ID NOs: 3, 7, 11, 15, or 19). For example, a mutant nucleic acid sequence can encode a mutant polypeptide sequence (e.g., truncated, functionally impaired), or a mutant nucleic acid sequence can encode a wild type polypeptide that is, for example, abnormally expressed (e.g., a reduced amount or improperly located polypeptide relative to the polypeptide encoded by the non-mutant nucleic acid sequence (e.g., a wild type nucleic acid sequence).

Methods of making a tobacco plant having a mutation are known in the art. Mutations can be random mutations or targeted mutations. For random mutagenesis, cells (e.g., Nicotiana tabacum cells) can be mutagenized using, for example, a chemical mutagen, ionizing radiation, or fast neutron bombardment (sec, e.g., Li et al., 2001, Plant J., 27:235-42). Representative chemical mutagens include, without limitation, nitrous acid, sodium azide, acridine orange, ethidium bromide, and ethyl methane sulfonate (EMS), while representative ionizing radiation includes, without limitation, x-rays, gamma rays, fast neutron irradiation, and UV irradiation. The dosage of the mutagenic chemical or radiation is determined experimentally for each type of plant tissue such that a mutation frequency is obtained that is below a threshold level characterized by lethality or reproductive sterility. The number of M₁ generation seed or the size of M₁ plant populations resulting from the mutagenic treatments are estimated based on the expected frequency of mutations. For targeted mutagenesis, representative technologies include TALEN (see, for example, Li et al., 2011, Nucleic Acids Res., 39(14):6315-25) or zinc-finger (see, for example, Wright et al., 2005, The Plant J., 44:693-705). Whether random or targeted, a mutation can be a point mutation, an insertion, a deletion, a substitution, or combinations thereof.

Conserved domains in polypeptides can be important for polypeptide function as well as cellular or subcellular location. As discussed herein, one or more nucleotides can be mutated to alter the expression and/or function of the encoded polypeptide, relative to the expression and/or function of the corresponding wild type polypeptide. It will be appreciated, for example, that a mutation in one or more highly conserved regions would likely alter polypeptide function, while a mutation outside of a conserved region would likely have little to no effect on polypeptide function. In addition, a mutation in a single nucleotide can create a stop codon, which would result in a truncated polypeptide and, depending on the extent of truncation, loss-of-function.

Preferably, a mutation in one of the nucleic acids disclosed herein results in reduced or even complete elimination of protein activity. Suitable types of mutations in a coding sequence include, without limitation, insertions of nucleotides, deletions of nucleotides, or transitions or transversions in the wild-type coding sequence. Mutations in the coding sequence can result in insertions of one or more amino acids, deletions of one or more amino acids, and/or non-conservative amino acid substitutions in the encoded polypeptide. In some cases, the coding sequence can include more than one mutation or more than one type of mutation.

Insertion or deletion of amino acids in a coding sequence, for example, can disrupt the conformation of the encoded polypeptide. Amino acid insertions or deletions also can disrupt sites important for recognition of a binding ligand or for activity of the polypeptide. It is known in the art that the insertion or deletion of a larger number of contiguous amino acids is more likely to render the gene product non-functional, compared to a smaller number of inserted or deleted amino acids. In addition, one or more mutations (e.g., a point mutation) can change the localization of the polypeptide in the cell, introduce a stop codon to produce a truncated polypeptide, or disrupt an active site or domain (e.g., a catalytic site or domain, a binding site or domain) within the polypeptide.

Non-conservative amino acid substitutions can replace an amino acid of one class with an amino acid of a different class. Non-conservative substitutions can make a substantial change in the charge or hydrophobicity of the polypeptide. Non-conservative amino acid substitutions can also make a substantial change in the bulk of the residue side chain, e.g., substituting an alanine residue for an isoleucine residue. Examples of non-conservative substitutions include a basic amino acid for a non-polar amino acid, or a polar amino acid for an acidic amino acid.

Transmembrane polypeptides contain particular sequences that determine where the polypeptide is localized within the cell. The target peptide sequences often are cleaved (e.g., by specific proteases that recognize a specific nucleotide motif) after the polypeptide is inserted into the membrane. By mutating the target sequence or a cleavage motif, the targeting of the polypeptide can be altered.

Following mutagenesis, M₀ plants are regenerated from the mutagenized cells and those plants, or a subsequent generation of that population (e.g., M₁, M₂, M₃, etc.), can be screened for a mutation in a sequence of interest (e.g., SEQ ID NOs: 1, 2, 5, 6, 9, 10, 13, 14, 17, and 18). Screening for plants carrying a mutation in a sequence of interest can be performed using methods routine in the art (e.g., hybridization, amplification, combinations thereof) or by evaluating the phenotype (e.g., an increase in yield or a decrease in the amount and/or rate of chlorophyll loss). Generally, the presence of a mutation in one or more of the nucleic acid sequences disclosed herein (e.g., SEQ ID NOs: 1, 2, 5, 6, 9, 10, 13, 14, 17, and 18) results in an increase in yield or a decrease in the amount and/or rate of chlorophyll loss compared to a corresponding plant (e.g., having the same varietal background) lacking the mutation.

An M₁ tobacco plant may be heterozygous for a mutant allele and exhibit a wild type phenotype. In such cases, at least a portion of the first generation of self-pollinated progeny of such a plant exhibits a wild type phenotype. Alternatively, an M₁ tobacco plant may have a mutant allele and exhibit a mutant phenotype. Such plants may be heterozygous and exhibit a mutant phenotype due to a phenomenon such as dominant negative suppression, despite the presence of the wild type allele, or such plants may be homozygous due to independently induced mutations in both alleles.

A tobacco plant carrying a mutant allele can be used in a plant breeding program to create novel and useful cultivars, lines, varieties and hybrids. Thus, in some embodiments, an M₁, M₂, M₃ or later generation tobacco plant containing at least one mutation is crossed with a second Nicotiana tabacum plant, and progeny of the cross are identified in which the mutation(s) is present. It will be appreciated that the second Nicotiana tabacum plant can be one of the species and varieties described herein. It will also be appreciated that the second Nicotiana tabacum plant can contain the same mutation as the plant to which it is crossed, a different mutation, or be wild type at the locus. Additionally or alternatively, a second tobacco line can exhibit a phenotypic trait such as, for example, disease resistance, high yield, high grade index, curability, curing quality, mechanical harvesting, holding ability, leaf quality, height, plant maturation (e.g., early maturing, early to medium maturing, medium maturing, medium to late maturing, or late maturing), stalk size (e.g., small, medium, or large), and/or leaf number per plant (e.g., a small (e.g., 5-10 leaves), medium (e.g., 11-15 leaves), or large (e.g., 16-21) number of leaves).

Breeding is carried out using known procedures. DNA fingerprinting, SNP or similar technologies may be used in a marker-assisted selection (MAS) breeding program to transfer or breed mutant alleles into other tobaccos, as described herein. Progeny of the cross can be screened for a mutation using methods described herein, and plants having a mutation in a nucleic acid sequence disclosed herein (e.g., SEQ ID NOs: 1, 2, 5, 6, 9, 10, 13, 14, 17, and 18) can be selected. For example, plants in the F₂ or backcross generations can be screened using a marker developed from a sequence described herein or a fragment thereof, using one of the techniques listed herein. Progeny plants also can be screened for an increase in yield or a decrease in the amount and/or rate of chlorophyll loss, and the desired plants, compared to a corresponding plant that lacks the mutation, can be selected. Plants identified as possessing the mutant allele and/or the mutant phenotype can be backcrossed or self-pollinated to create a second population to be screened. Backcrossing or other breeding procedures can be repeated until the desired phenotype of the recurrent parent is recovered.

Successful crosses yield F₁ plants that are fertile and that can be backcrossed with one of the parents if desired. In some embodiments, a plant population in the F₂ generation is screened for the mutation or variant gene expression using standard methods (e.g., PCR with primers based upon the nucleic acid sequences disclosed herein). Selected plants are then crossed with one of the parents and the first backcross (BC₁) generation plants are self-pollinated to produce a BC₁F₂ population that is again screened for variant gene expression. The process of backcrossing, self-pollination, and screening is repeated, for example, at least four times until the final screening produces a plant that is fertile and reasonably similar to the recurrent parent. This plant, if desired, is self-pollinated and the progeny are subsequently screened again to confirm that the plant contains the mutation and exhibits variant gene expression. Breeder's seed of the selected plant can be produced using standard methods including, for example, field testing, confirmation of the null condition, and/or chemical analyses of leaf to determine chlorophyll loss.

The result of a plant breeding program using the mutant tobacco plants described herein are novel and useful cultivars, varieties, lines, and hybrids. As used herein, the term “variety” refers to a population of plants that share constant characteristics which separate them from other plants of the same species. A variety is often, although not always, sold commercially. While possessing one or more distinctive traits, a variety is further characterized by a very small overall variation between individual with that variety. A “pure line” variety may be created by several generations of self-pollination and selection, or vegetative propagation from a single parent using tissue or cell culture techniques. A “line,” as distinguished from a variety, most often denotes a group of plants used non-commercially, for example, in plant research. A line typically displays little overall variation between individuals for one or more traits of interest, although there may be some variation between individuals for other traits.

A variety can be essentially derived from another line or variety. As defined by the International Convention for the Protection of New Varieties of Plants (Dec. 2, 1961, as revised at Geneva on Nov. 10, 1972, On Oct. 23, 1978, and on Mar. 19, 1991), a variety is “essentially derived” from an initial variety if: a) it is predominantly derived from the initial variety, or from a variety that is predominantly derived from the initial variety, while retaining the expression of the essential characteristics that result from the genotype or combination of genotypes of the initial variety; b) it is clearly distinguishable from the initial variety; and c) except for the differences which result from the act of derivation, it confirms to the initial variety in the expression of the essential characteristics that result from the genotype or combination of genotypes of the initial variety. Essentially derived varieties can be obtained, for example, by the selection of a natural or induced mutant, a somaclonal variant, a variant individual plant from the initial variety, backcrossing, or transformation.

Tobacco hybrids can be produced by preventing self-pollination of female parent plants (i.e., seed parents) of a first variety, permitting pollen from male parent plants of a second variety to fertilize the female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by cytoplasmic male sterility (CMS), nuclear male sterility, genetic male sterility, molecular male sterility wherein a transgene inhibits microsporogenesis and/or pollen formation, or self-incompatibility. Female parent plants containing CMS are particularly useful. In embodiments in which the female parent plants are CMS, the male parent plants typically contain a fertility restorer gene to ensure that the F₁ hybrids are fertile. In other embodiments in which the female parents are CMS, male parents can be used that do not contain a fertility restorer. F₁ hybrids produced from such parents are male sterile. Male sterile hybrid seed can be interplanted with male fertile seed to provide pollen for seed-set on the resulting male sterile plants.

Varieties, lines and cultivars described herein can be used to form single-cross tobacco F₁ hybrids. In such embodiments, the plants of the parent varieties can be grown as substantially homogeneous adjoining populations to facilitate natural cross-pollination from the male parent plants to the female parent plants. The F₂ seed formed on the female parent plants is selectively harvested by conventional means. One also can grow the two parent plant varieties in bulk and harvest a blend of F₁ hybrid seed formed on the female parent and seed formed upon the male parent as the result of self-pollination. Alternatively, three-way crosses can be carried out wherein a single-cross F₁ hybrid is used as a female parent and is crossed with a different male parent. As another alternative, double-cross hybrids can be created wherein the F₁ progeny of two different single-crosses are themselves crossed. Self-incompatibility can be used to particular advantage to prevent self-pollination of female parents when forming a double-cross hybrid.

The tobacco plants used in the methods described herein can be a Burley type, a dark type, a flue-cured type, a Maryland type, or an Oriental type. The tobacco plants used in the methods described herein typically are from N. tabacum, and can be from any number of N. tabacum varieties. A variety can be BU 64, CC 101, CC 200, CC 13, CC 27, CC 33, CC 35,CC 37, CC 65, CC 67, CC 301, CC 400, CC 500, CC 600, CC 700, CC 800, CC 900, CC 1063, Coker 176, Coker 319, Coker 371 Gold, Coker 48, CU 263, DF911, Galpao tobacco, GL 26H, GL 338, GL 350, GL 395, GL 600, GL 737, GL 939, GL 973, GF 157, GF 318, RJR 901, HB 04P, K 149, K 326, K 346, K 358, K394, K 399, K 730, NC 196, NC 37NF, NC 471, NC 55, NC 92, NC2326, NC 95, NC 925, PVH 1118, PVH 1452, PVH 2110, PVH 2254, PVH 2275, VA 116, VA 119, KDH 959, KT 200, KT204LC, KY 10, KY 14, KY 160, KY 17, KY 171, KY 907, KY907LC, KTY14 x L8 LC, Little Crittenden, McNair 373, McNair 944, msKY 14xL8, Narrow Leaf Madole, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, ‘Perique’ tobacco, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN90LC, TN 97, TN97LC, TN D94, TN D950, TR (Tom Rosson) Madole, VA 309, or VA359.

In addition to mutation, another way in which nitrogen utilization can be increased is by using inhibitory RNAs (e.g., RNAi). Therefore, transgenic tobacco plants are provided that contain a transgene encoding at least one RNAi molecule, which, when expressed, silences at least one of the endogenous nucleic acids described herein (e.g., SEQ ID NOs: 1, 2, 5, 6, 9, 10, 13, 14, 17, and 18). As described herein, leaf from such transgenic plants exhibit an increase in yield (e.g., compared to leaf from a plant lacking or not expressing the RNAi). In addition, leaf from such transgenic plants exhibit a decrease in the amount and/or rate of chlorophyll loss (e.g., compared to leaf from a plant lacking or not expressing the RNAi).

RNAi technology is known in the art and is a very effective form of post-transcriptional gene silencing. RNAi molecules typically contain a nucleotide sequence (e.g., from about 18 nucleotides in length (e.g., about 19 or 20 nucleotides in length) up to about 700 nucleotides in length) that is complementary to the target gene in both the sense and antisense orientations. The sense and antisense strands can be connected by a short “loop” sequence (e.g., about 5 nucleotides in length up to about 800 nucleotides in length) and expressed in a single transcript, or the sense and antisense strands can be delivered to and expressed in the target cells on separate vectors or constructs. A number of companies offer RNAi design and synthesis services (e.g., Life Technologies, Applied Biosystems).

The RNAi molecule typically is at least 25 nucleotides in length and has at least 91% sequence identity (e.g., at least 95%, 96%, 97%, 98% or 99% sequence identity) to one of the nucleic acid sequences disclosed herein (e.g., SEQ ID NOs: 1, 2, 5, 6, 9, 10, 13, 14, 17, and 18) or hybridizes under stringent conditions to one of the nucleic acid sequences disclosed herein (e.g., SEQ ID NOs: 1, 2, 5, 6, 9, 10, 13, 14, 17, and 18). The RNAi molecule can be expressed using a plant expression vector, and hybridization under stringent conditions is described above. Representative RNAi molecules to a number of the novel sequences described herein are provided in SEQ ID NOs: 4, 8, 12, 16, or 20.

Methods of introducing a nucleic acid (e.g., a heterologous nucleic acid) into plant cells are known in the art and include, for example, particle bombardment, Agrobacterium-mediated transformation, microinjection, polyethylene glycol-mediated transformation (e.g., of protoplasts, see, for example, Yoo et al. (2007, Nature Protocols, 2(7):1565-72)), liposome-mediated DNA uptake, or electroporation. Following transformation, the transgenic plant cells can be regenerated into transgenic tobacco plants. As described herein, expression of the transgene results in plants that exhibit an increase in yield or a decrease in the amount and/or rate of chlorophyll loss relative to a plant not expressing the transgene. The regenerated transgenic plants can be screened for an increase in yield or a decrease in the amount and/or rate of chlorophyll loss, compared to the amount in a corresponding non-transgenic plant. In addition, a regenerated transgenic plant having an increase in yield or a decrease in the amount and/or rate of chlorophyll loss can be selected for use in, for example, a breeding program as discussed herein.

It would be understood by the skilled artisan that yield, with respect to tobacco, refers to leaf dry matter, leaf number, leaf surface area, leaf thickness, and/or total dry matter. As used herein, an increase in yield refers to an increase (e.g., a statistically significant increase) in the yield of tobacco plants by at least about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to similarly-grown tobacco plants lacking the mutation or the transgene or relative to TN90. As used herein, statistical significance refers to a p-value of less than 0.05, e.g., a p-value of less than 0.025 or a p-value of less than 0.01, using an appropriate measure of statistical significance, e.g., a one-tailed two sample t-test.

Loss of chlorophyll due to its breakdown is a regular process that occurs in leaves during senescence. See, for example, Hortensteiner & Krautler (2011, Biochim. Biophys. Acta, 1807:977-88). As used herein, a decrease in the amount and/or rate of chlorophyll loss refers to a decrease (e.g., a statistically significant decrease) in the amount and/or rate of chlorophyll loss in tobacco plants (e.g., tobacco leaf) by about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to similarly-grown tobacco lacking the mutation or the transgene or relative to TN90. As used herein, statistical significance refers to a p-value of less than 0.05, e.g., a p-value of less than 0.025 or a p-value of less than 0.01, using an appropriate measure of statistical significance, e.g., a one-tailed two sample t-test. Chlorophyll, and, therefore, the loss of chlorophyll, can be measured by using a SPAD meter, which measures the specific absorbance of chlorophyll, or by measuring the green coloration in the leaf by image analysis algorithms.

Nucleic acids that confer traits such as herbicide resistance (sometimes referred to as herbicide tolerance), insect resistance, or stress tolerance, can also be present in the tobacco plants described herein. Genes conferring resistance to an herbicide that inhibits the growing point or meristem, such as an imidazolinone or a sulfonylurea, can be suitable. Exemplary genes in this category encode mutant ALS and AHAS enzymes as described, for example, in U.S. Pat. Nos. 5,767,366 and 5,928,937. U.S. Pat. Nos. 4,761,373 and 5,013,659 are directed to plants resistant to various imidazolinone or sulfonamide herbicides. U.S. Pat. No. 4,975,374 relates to plant cells and plants containing a gene encoding a mutant glutamine synthetase (GS), which is resistant to inhibition by herbicides that are known to inhibit GS, e.g. phosphinothricin and methionine sulfoximine. U.S. Pat. No. 5,162,602 discloses plants resistant to inhibition by cyclohexanedione and aryloxyphenoxypropanoic acid herbicides.

Genes for resistance to glyphosate also are suitable. See, for example, U.S. Pat. Nos. 4,940,835 and 4,769,061. Such genes can confer resistance to glyphosate herbicidal compositions, including, without limitation, glyphosate salts such as the trimethylsulphonium salt, the isopropylamine salt, the sodium salt, the potassium salt and the ammonium salt. See, e.g., U.S. Pat. Nos. 6,451,735 and 6,451,732. Genes for resistance to phosphono compounds such as glufosinate ammonium or phosphinothricin, and pyridinoxy or phenoxy propionic acids and cyclohexones also are suitable. See, e.g., U.S. Pat. Nos. 5,879,903; 5,276,268; and 5,561,236; and European Application No. 0 242 246.

Other suitable herbicides include those that inhibit photosynthesis, such as a triazine and a benzonitrile (nitrilase). See U.S. Pat. No. 4,810,648. Other suitable herbicides include 2,2-dichloropropionic acid, sethoxydim, haloxyfop, imidazolinone herbicides, sulfonylurea herbicides, triazolopyrimidine herbicides, s-triazine herbicides and bromoxynil. Also suitable are herbicides that confer resistance to a protox enzyme. See, e.g., U.S. Pat. No. 6,084,155 and US 2001/0016956.

A number of genes are available that confer resistance to insects, for example, insects in the order Lepidoptera. Exemplary genes include those that encode truncated Cry1A(b) and Cry1A(c) toxins. See, e.g., genes described in U.S. Pat. Nos. 5,545,565; 6,166,302; and 5,164,180. See also, Vaeck et al., 1997, Nature, 328:33-37 and Fischhoff et al., 1987, Nature Biotechnology, 5:807-813. Particularly useful are genes encoding toxins that exhibit insecticidal activity against Manduca sexta (tobacco hornworm); Heliothis virescens Fabricius (tobacco budworm) and/or S. litura Fabricius (tobacco cutworm).

In addition to mutation and RNAi, another way in which nitrogen utilization can be increased is by overexpressing one or more nucleic acid molecules. The sequences described herein can be overexpressed in plants to an increase in yield or a decrease in the amount and/or rate of chlorophyll loss. Therefore, transgenic tobacco plants, or leaf from such plants, are provided that are transformed with a nucleic acid molecule described herein (e.g., SEQ ID NOs: 1, 2, 5, 6, 9, 10, 13, 14, 17, and 18) or a functional fragment thereof under control of a promoter that is able to drive expression in plants (e.g., a plant promoter). As discussed herein, a nucleic acid molecule used in a plant expression vector can have a different sequence than a sequence described herein, which can be expressed as a percent sequence identity (e.g., relative to SEQ ID NOs: 1, 2, 5, 6, 9, 10, 13, 14, 17, and 18) or based on the conditions under which the sequence hybridizes to SEQ ID NOs: 1, 2, 5, 6, 9, 10, 13, 14, 17, and 18.

As an alternative to using a full-length sequence, a portion of the sequence can be used that encodes a polypeptide fragment having the desired functionality (referred to herein as a “functional fragment”). When used with respect to nucleic acids, it would be appreciated that it is not the nucleic acid fragment that possesses functionality but the encoded polypeptide fragment. Based on the disclosure herein, one of skill in the art can predict or readily identify the portion(s) of a polypeptide (e.g., one or more domains) that may impart the desired functionality.

Following transformation, the transgenic tobacco cells can be regenerated into transgenic tobacco plants. The regenerated tobacco plants can be screened for an increase in yield or a decrease in the amount and/or rate of chlorophyll loss, and plants having an increase in yield or a decrease in the amount and/or rate of chlorophyll loss, compared to the amount in a corresponding non-transgenic plant, can be selected and used, for example, in a breeding program as discussed herein. Expression of the nucleic acid molecule or a functional fragment thereof may result in tobacco plants that exhibit an increase in yield or a decrease in the amount and/or rate of chlorophyll loss compared to a tobacco plant that does not express the nucleic acid molecule or functional fragment thereof. Nucleic acids conferring herbicide resistance, insect resistance, or stress tolerance, can also be introduced into such tobacco plants.

### Tobacco Products and Methods of Making

The methods described herein allow for tobacco plants to be produced that exhibit an increase in yield or a decrease in the amount and/or rate of chlorophyll loss. As described herein, such methods can include mutagenesis (e.g., random or targeted) or the production of transgenic plants (using, e.g., RNAi or overexpression).

Leaf from such tobacco (e.g., exhibiting an increase in yield or a decrease in the amount and/or rate of chlorophyll loss) can be cured, aged, conditioned, and/or fermented. Methods of curing tobacco are well known and include, for example, air curing, fire curing, flue curing and sun curing. Aging also is known and is typically carried out in a wooden drum (e.g., a hogshead) or cardboard cartons in compressed conditions for several years (e.g., 2 to 5 years), at a moisture content of from about 10% to about 25% (see, for example, U.S. Pat. No. 4,516,590 and 5,372,149). Conditioning includes, for example, a heating, sweating or pasteurization step as described in US 2004/0118422 or US 2005/0178398, while fermenting typically is characterized by high initial moisture content, heat generation, and a 10 to 20% loss of dry weight. See, e.g., U.S. Pat. Nos. 4,528,993, 4,660,577, 4,848,373 and 5,372,149. The tobacco also can be further processed (e.g., cut, expanded, blended, milled or comminuted), if desired, and used in a tobacco product.

Tobacco products are known in the art and include any product made or derived from tobacco that is intended for adult human consumption, including any component, part, or accessory of a tobacco product. Representative tobacco products include, without limitation, cigarettes, smokeless tobacco products, tobacco-derived nicotine products, cigarillos, non-ventilated recess filter cigarettes, vented recess filter cigarettes, cigars, snuff, e-vapor products, pipe tobacco, cigar tobacco, cigarette tobacco, chewing tobacco, leaf tobacco, shredded tobacco, and cut tobacco. Representative smokeless tobacco products include, for example, chewing tobacco, snus, pouches, films, tablets, coated dowels, rods, and the like. Representative cigarettes and other smoking articles include, for example, smoking articles that include filter elements or rod elements, where the rod element of a smokeable material can include cured tobacco within a tobacco blend. In addition to the reduced-alkaloid tobacco described herein, tobacco products also can include other ingredients such as, without limitation, binders, plasticizers, stabilizers, and/or flavorings. See, for example, US 2005/0244521, US 2006/0191548, US 2012/0024301, US 2012/0031414, and US 2012/0031416 for examples of tobacco products.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Tobacco with Decreased Nitrogen Requirements

Burley tobacco is considered to be nitrogen inefficient whereas a related type of tobacco, Maryland, is grown with much less fertilizer. This fact was utilized to determine the cause of nitrogen use efficiency in Maryland tobacco and to translate these findings to burley tobacco. Gene expression and metabolite analysis of TN90 and Maryland tobacco were also used to find metabolic pathways for possible manipulation in burley tobacco and to find molecular markers for the Maryland phenotype. These markers also can be used to compare burley tobacco with other nitrogen use efficient tobacco varieties and Nicotiana species for future breeding stocks.

### Example 2—Analysis of Gene Expression Levels

In order to determine the genetic basis for the nitrogen utilization efficiency observed in Maryland tobacco, gene expression in roots and leaves of both Md609 and TN90 tobacco were analyzed. Briefly, seedlings of both types of tobacco were grown without added nitrogen for 4 weeks. Plants were then grown on 25% nitrogen for up to 8 weeks with samples of roots and leaves being taken at multiple time points. RNA was extracted from both the roots and leaves and sequenced on an Illumina Next-Gen sequencing platform. RNA-seq gene expression analysis was done using the previously obtained genome information from the sequencing of TN90. Gene expression analysis identified 5 genes that would be predicted to affect the nitrogen use efficiency in burley tobacco. These genes are listed, along with their expression level in TN90 and Maryland tobacco, in Tables 1, 2 and 3.

TABLE 1
Sequences

| | | Coding | Gene | Protein | Overexpression/ |
|---|---|---|---|---|---|
| Gene | | Sequence | Sequence | Sequence | RNAi |
| Designation | Homology | (SEQ ID NO) | (SEQ ID NO) | (SEQ ID NO) | (SEQ ID NO) |
| g80368_ | dof zinc finger | 1 | 2 | 3 | 4 |
| Scaffold0001709 | | | | | |
| g75333_ | protein TIC | 5 | 6 | 7 | 8 |
| Scaffold0001539 | 22-like | | | | |
| g1645_ | glutamate | 9 | 10 | 11 | 12 |
| Scaffold0000011 | decarboxylase | | | | |
| g173910_ | mads-box TF | 13 | 14 | 15 | 16 |
| Scaffold0007958 | 27-like | | | | |
| g112803_ | PKHD-type | 17 | 18 | 19 | 20 |
| Scaffold0003083 | hydroxylase | | | | |

TABLE 2
Leaf Expression at 25% Fertilization Rate

| | | TN90 | | | | | | | |
|---|---|---|---|---|---|---|---|---|---|
| Gene | | Time | TN90 | TN90 | TN90 | Md609 | Md609 | Md609 | Md609 |
| Designation | Homology | 0 | 1 wk | 4 wks | 8 wks | Time 0 | 1 wk | 4 wks | 8 wks |
| g112803_ | PKHD-type | 6.45 | 10.77 | 11.10 | 7.36 | 0.00 | 0.00 | 0.00 | 0.00 |
| Scaffold0003083 | hydroxylase | | | | | | | | |
| g1645_ | glutamate | 27.54 | 20.40 | 19.18 | 22.85 | 2.31 | 2.87 | 0.87 | 1.22 |
| Scaffold0000011 | decarboxylase | | | | | | | | |
| g173910_ | mads-box TF | 20.37 | 21.44 | 20.47 | 6.45 | 50.99 | 53.41 | 72.87 | 21.19 |
| Scaffold0007958 | 27-like | | | | | | | | |
| g75333_ | protein TIC | 3.21 | 5.27 | 3.53 | 2.51 | 29.60 | 109.12 | 39.51 | 35.59 |
| Scaffold0001539 | 22-like | | | | | | | | |
| g80368_ | dof zinc finger | 2.04 | 2.49 | 6.20 | 1.00 | 2.89 | 0.34 | 2.74 | 1.29 |
| Scaffold0001709 | | | | | | | | | |

TABLE 3
Root Expression at 25% Fertilization Rate

| | | TN90 | | | | | | | |
|---|---|---|---|---|---|---|---|---|---|
| | | Time | TN90 | TN90 | TN90 | Md609 | Md609 | Md609 | Md609 |
| Gene Designation | Homology | 0 | 1 wk | 4 wks | 8 wks | Time 0 | 1 wk | 4 wks | 8 wks |
| g112803_ | PKHD-type | 9.78 | 11.55 | 8.74 | 13.97 | 0.00 | 0.00 | 0.00 | 0.00 |
| Scaffold0003083 | hydroxylase | | | | | | | | |
| g1645_ | glutamate | 37.05 | 21.74 | 24.89 | 26.26 | 2.02 | 1.10 | 0.75 | 0.29 |
| Scaffold0000011 | decarboxylase | | | | | | | | |
| g173910_ | mads-box TF | 14.36 | 8.35 | 10.01 | 16.33 | 69.64 | 13.40 | 34.55 | 17.25 |
| Scaffold0007958 | 27-like | | | | | | | | |
| g75333_ | protein TIC | 2.59 | 2.52 | 2.83 | 4.45 | 29.46 | 57.61 | 56.56 | 66.56 |
| Scaffold0001539 | 22-like | | | | | | | | |
| g80368_ | dof zinc finger | 23.19 | 13.24 | 16.72 | 17.72 | 55.43 | 13.10 | 44.41 | 20.45 |
| Scaffold0001709 | | | | | | | | | |

### Example 3—Metabolite Profiling for Nitrogen Use Efficiency Marker Development

Metabolite analysis was done in parallel to determine the flux of nitrogen through the metabolic pathways and relate this to the gene expression data. Shotgun metabolomics using a combination of UHPLC-MS/MS and GC-MS was done by Metabolon Inc. Relative levels of 616 compounds from leaves and 771 compounds from roots were evaluated. Only a subset of these compounds could be identified with high confidence (359 compounds from leaf and 449 compounds from root). Relevant metabolites were used to validate the gene expression differences and to determine possible markers for the Maryland phenotype.

The metabolite profiles that were generated were used to develop a panel of molecular markers that could be used to define the Maryland phenotype and for further quantitative trait loci discovery. These markers mostly relate to nitrogen assimilation and storage. One set of markers was determined to be applicable to the nitrogen use efficiency of Maryland tobacco at reduced fertilization rates. A second set of markers was found to be indicative of nitrogen toxicity in Maryland tobacco at normal Burley levels of fertilization. The list of markers is shown in Table 4 with their relative values between TN90 and Maryland after nitrogen starvation conditions. Table 5 shows metabolic markers that may be indicative of nitrogen toxicity in Maryland tobacco at the normal burley fertilization rate.

TABLE 4
Metabolites Associated with Nitrogen Use
Efficiency at Low Nitrogen Levels

| Biochemical Name | MD609/TN90 |
|---|---|
| Spermine | 0.13 |
| Lysine | 0.26 |
| Isoleucine | 0.31 |
| Valine | 0.37 |
| Phenylalanine | 0.38 |
| Tyrosine | 0.38 |
| Spermidine | 0.43 |
| Asparagine | 0.47 |
| Histidine | 0.47 |
| Leucine | 0.48 |
| Arginine | 0.76 |

TABLE 5
Metabolites Associated with Nitrogen
Toxicity at High Nitrogen Levels

| Biochemical Name | MD609/TN90 |
|---|---|
| Raffinose | 33.02 |
| Glycerophosphorylcholine (GPC) | 62.74 |
| Nicotianamine | 3.25 |
| Allantoic acid | 12.74 |
| Allantoin | 1.91 |

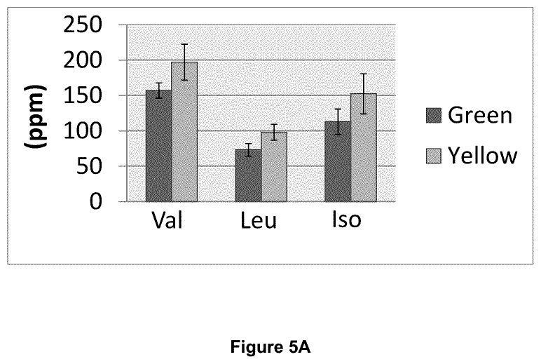

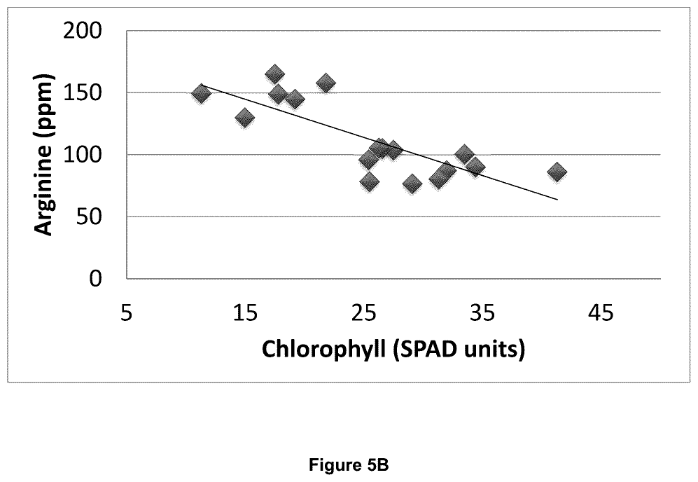

Metabolite markers were also measured in a segregating F2 population resulting from the cross between TN90 and Maryland 609. Plants were grown in the field using 45 lbs/ac of nitrogen. Phenotypic data was collected from the population four weeks after topping (at harvest) including chlorophyll content, yield, stalk diameter, and height. Amino acid content was measured from freeze dried samples and correlated to the relevant phenotypic data. Representative data is shown in FIG. 5A and 5B.

### Example 4—RNAi Line Development, Plasmid Construction and Agrobacterium Transformation

To investigate the function of the candidate genes, transgenic plants were generated expressing either the full length coding sequence of overexpression gene candidates or an RNAi sequence for downregulation of the gene candidate. An expression vector, p45-2-7 (see, for example, SEQ ID NO:57 in US 2015/0173319), was used, which has a CsVMV promoter and a NOS terminator, as well as a cassette having a kanamycin selection marker (NPT II) under direction of the actin2 promoter and a NOS terminator. The nucleic acid constructs carrying the transgenes of interest were introduced into tobacco leaf discs using an Agrobacterium transformation approach. See, for example, Mayo et al., 2006, Nat Protoc., 1(3):1105-11 and Horsch et al., 1985, Science 227:1229-1231.

Briefly, ascetical tobacco plants (Tennessee 90 (TN90)) were grown from magenta boxes, and leaves discs were cut into 15×150 mm plates. Agrobacterium tumefaciens containing the target plasmid were collected by centrifugation of 20 ml cell suspension in 50 ml centrifuge tube at 3500 rpm for 10 minutes. Supernatant was removed and Agrobacterium cell pellet was resuspended in 40 ml liquid resuspension medium. About 25 ml of the solution was transferred to each 15×100 mm Petri plates. In those 15×150 mm plates, tobacco leaves, avoiding the midrib, were cut into 0.6 cm disk. Leaf disks were placed upside down, a thin layer of MS/B5 liquid resuspension medium was added, and slices were made with a #15 razor blade. The leaf discs were poked uniformly with a fine point needle. Eight disks were placed, upside down, in each regeneration plate (15×100 mm). Agrobacterium tumefaciens suspension was added and the leaf discs were incubated for 10 minutes.

Leaf disks were transferred to co-cultivation plates (1/2 MS medium) and disks were placed upside down in contact with filter paper overlaid on the co-cultivation TOM medium (MS medium with 20 g sucrose/L; 1 mg/L IAA and 2.5 mg/L BAP). The plate was sealed with parafilm and labeled appropriately. Plates were incubated in dim light (60-80 mE/ms) and 18/6 photoperiods at 24° C. for three days. Leaf disks were transferred to regeneration/selection TOM K medium plates (TOM medium with 300 mg/1 Kanamycin) and subculture bi-weekly to the same fresh medium in dim light at 24° C. until shoots become excisable. Shoots from leaves were removed with forceps and inserted in MS basal medium with 100 mg/L kanamycin at 24° C. and 18/6 photoperiods with light intensity of 6080 mE/ms for rooting.

When plantlets with both shoots and roots grew large enough (e.g., reach over half of a GA7 box), they were transferred to soil for acclimatization. During the transfer, the gel was washed from the root tissue with tap water. Established seedlings were transferred to the greenhouse for further analysis and to set seed.

### Example 5—Random Mutagenesis and Characterization of Mutants

One gram (approximately 10,000 seeds) of Tennessee 90 tobacco (TN90) converter seed was washed in 0.1% Tween® for fifteen minutes and then soaked in 30 ml of ddH₂O for two hours. One hundred fifty (150) μl of 0.5% EMS (Sigma, Catalogue No. M-0880) was then mixed into the seed/ddH₂O solution and incubated for 8-12 hours (rotating at 30 rpm) under a hood at room temperature (RT; approximately 20° C.). The liquid then was removed from the seeds and the liquid was mixed into 1 M NaOH overnight for decontamination and disposal. The seeds were then washed twice with 100 ml ddH₂O for 2-4 hours. The washed seeds were then suspended in 0.1% agar: water solution.

The EMS-treated seeds in the agar solution were evenly spread onto water-soaked Carolina's Choice Tobacco Mix™ (Carolina Soil Company, Kinston, NC) in flats at ˜2000 seeds/flat. The flats were then covered with plastic wrap and placed in a growth chamber. Once the seedlings emerged from the soil, the plastic wrap was punctured to allow humidity to decline gradually. The plastic wrap was completely removed after two weeks. Flats were moved to a greenhouse and fertilized with NPK fertilizer. The seedlings were plugged into a float tray and grown until transplanting size. The plants were transplanted into a field. During growth, the plants were self-pollinated to form M₁ seeds. At the mature stage, five capsules were harvested from each plant and individual designations were given to the set of seeds from each plant. This formed the M₁ population.

A composite of M₁ seed from each M₀ plant was grown, and leaves from M₁ plants were collected and DNA extracted. Target genes were amplified and sequenced for mutation identification.

### Example 6—Screening Plants for Modulation of Nitrogen Use Efficiency

There are multiple ways for plants to exhibit increased nitrogen use efficiency. The first is nitrogen uptake, the second is nitrogen assimilation, and the third is nitrogen storage. The integration of these three pathways allows for the most efficient use of nitrogen. The regulation of these three stages can occur in response to light. Under normal field conditions, fertilizer is applied preplant with another amount applied at layby stage. This differs from greenhouse grown plants in that the plants are constantly fed nitrogen through an “Ebb and Flow” system. We have developed a two-step protocol for screening of nitrogen use efficiency. First seedlings are grown under zero nitrogen conditions for 3 weeks and the rate of chlorophyll loss and growth is measured. Selected populations are then grown with a step down fertilization protocol to mimic the depletion of nitrogen in a greenhouse setting.

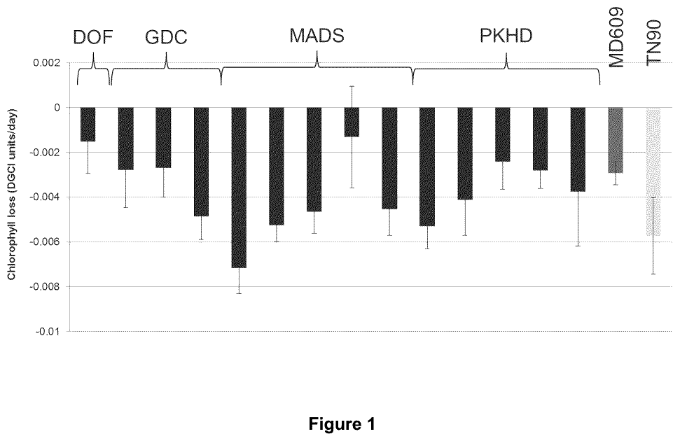

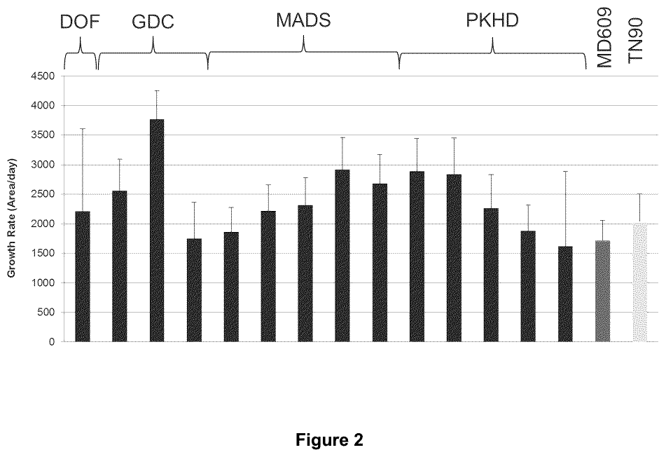

Transgenic and mutant tobacco plants are grown in a greenhouse in 21 cell growth trays. Measurements of chlorophyll loss and plant growth are taken by standard RGB based imaging and image analysis. Representative data is shown in FIGS. 1 and 2. Glutamate decarboxylase RNAi (GDC-RNAi) and MADS-box RNAi expressing lines show reduced chlorophyll loss after nitrogen starvation and increased growth rate relative to burley control lines.

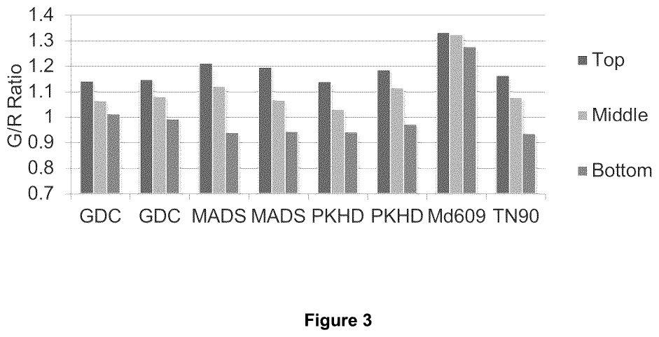

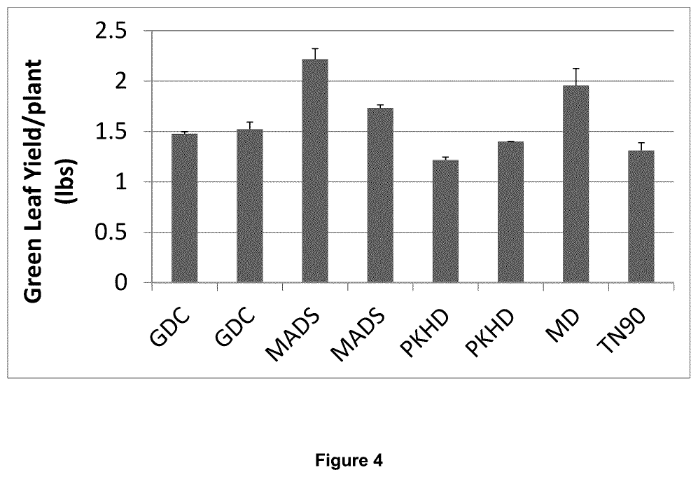

Transgenic and mutant tobacco plants are grown in a greenhouse under field-like conditions in Carolina's Choice Tobacco Mix (Carolina Soil Co., Kinston, NC) in 10 inch pots. Fertilization was reduced over time to mimic the reduction of nitrogen over time in the field setting. Measurements of chlorophyll content, and leaf weight were taken. Only one population was used per table to reduce population effects. Representative results from these studies are shown in FIGS. 3 and 4. Reduction in gene expression of the GDC gene resulted in a 12-16% increase in yield relative to the burley control. Reduction in MADS-box gene expression resulted in a 32-69% increase in yield. The Maryland control yielded 49% higher than the burley control. The distribution of chlorophyll throughout the plant was also different. GDC RNAi plants exhibited higher levels of retained chlorophyll in the lower leaves while the upper leaves contained comparable levels of chlorophyll. This suggests a decrease in the amount of redistributed nitrogen from the lower leaves.

### Example 7—Introgression of the Maryland Alleles by Breeding

An example of another way to introduce gene expression differences into burley tobacco to increase nitrogen use efficiency is by breeding. The genes of interest described above can be used as markers in a conventional breeding program to select for burley plants containing the genes from Maryland tobacco that confer nitrogen use efficiency.

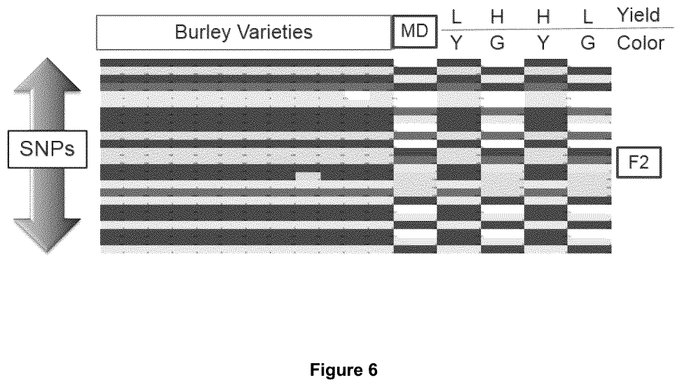

The Axiom Chip, containing 170,000 SNPs, was used to identify SNPs related to nitrogen utilization efficiency (NUE). FIG. 6 shows a graphical representation of the genotypes among Burley varieties and a Maryland variety and a segregating F2 population. The SNPs identified correlate with the Maryland trait of slower chlorophyll loss as the F2 plants that contain the Maryland alleles were still green at harvest. The SNPs identified can be used to track “green” plants (i.e., plants that have an amount and/or rate of chlorophyll loss similar to that of Maryland tobaccos), yield, and other characteristics of nitrogen utilization efficiency in tobacco.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1-18. (Canceled)

19. A tobacco plant comprising an induced mutation in a MADS-box transcription factor (TF) 27-like gene, wherein the MADS-box TF 27-like gene comprises an allele comprising a nucleic acid sequence of SEQ ID NO: 13 or SEQ ID NO: 14, wherein the induced mutation encodes an introduced stop codon that results in a truncated MADS-box TF 27-like polypeptide, and wherein the induced mutation causes a loss-of-function of the truncated MADS-box TF 27-like polypeptide.

20. The tobacco plant of claim 19, wherein the induced mutation comprises a point mutation or a substitution as compared to the nucleic acid sequence of SEQ ID NO: 13 or SEQ ID NO: 14.

21. The tobacco plant of claim 19, wherein the induced mutation comprises an insertion or a deletion as compared to the nucleic acid sequence of SEQ ID NO: 13 or SEQ ID NO: 14.

22. The tobacco plant of claim 19, wherein the tobacco plant exhibits an increase in yield relative to a tobacco plant lacking the induced mutation.

23. The tobacco plant of claim 19, wherein the tobacco plant exhibits decreased or slowed chlorophyll loss relative to a tobacco plant lacking the induced mutation.

24. The tobacco plant of claim 19, wherein the tobacco plant is of a type selected from the group consisting of a Burley type, a dark type, a flue-cured type, a Maryland type, and an Oriental type.

25. A tobacco seed from the tobacco plant of claim 19, wherein the tobacco seed comprises the induced mutation.

26. Cured tobacco leaf from the tobacco plant of claim 19.

27. The cured tobacco leaf of claim 26, wherein the cured tobacco leaf is selected from the group consisting of an air-cured tobacco leaf, a fire-cured tobacco leaf, and a sun-cured tobacco leaf.

28. A tobacco product comprising the cured tobacco leaf of claim 26.

29. The tobacco product of claim 28, wherein the tobacco product is selected from the group consisting of a cigarette, a cigar, a cigarillo, and a smokeless tobacco product.

30. The tobacco product of claim 29, wherein the smokeless tobacco product is selected from the group consisting of chewing tobacco and snus.

31. The tobacco product of claim 28, wherein the tobacco product comprises one or more ingredients selected from the group consisting of a binder, a plasticizer, a stabilizer, and a flavoring.

32. Fermented tobacco leaf from the tobacco plant of claim 19.

33. A method for producing a tobacco plant, the method comprising:
(a) inducing mutagenesis in tobacco cells to produce mutagenized cells;
(b) obtaining one or more tobacco plants from the mutagenized cells; and
(c) identifying at least one tobacco plant obtained in step (b) that comprises an induced mutation an endogenous nucleic acid encoding a MADS-box transcription factor (TF) 27-like polypeptide, wherein the endogenous nucleic acid comprises the nucleic acid sequence of SEQ ID NO. 13 or SEQ ID NO: 14, wherein the induced mutation comprises an introduced stop codon that results in a truncated a MADS-box TF 27-like polypeptide, and wherein the induced mutation causes a loss-of-function of the MADS-box TF 27-like gene polypeptide.

34. The method of claim 28, wherein the at least one tobacco plant identified in step (c) exhibits an increase in yield relative to a plant lacking the induced mutation.

35. The method of claim 28, wherein the at least one tobacco plant identified in step (c) exhibits decreased or slowed chlorophyll loss relative to a plant lacking the induced mutation.

36. The method of claim 28, wherein the mutagenesis comprises the use of an agent selected from the group consisting of a chemical mutagen, ionizing radiation, a TALEN, a CRISPR/Cas9, and a zinc-finger nuclease.

37. The method of claim 28, wherein the induced mutation comprises a point mutation or a substitution as compared to the nucleic acid sequence of SEQ ID NO: 13 or SEQ ID NO: 14.

38. The method of claim 28, wherein the induced mutation comprises an insertion or a deletion as compared to the nucleic acid sequence of SEQ ID NO: 13 or SEQ ID NO: 14.


SEQUENCE LISTING (20 sequences)

SEQ ID NO: 1 - DNA, 738 bp, Nicotiana tabacum
  source 1..738: mol_type = genomic DNA
atgccttcagacgtaagtgaacgaaaagctaccaaacaacagcaaggagctgcaccggcaccggagccggagcatcttccatgtccacgctgtgattccaccaacactaaattctgctactacaacaactacaacttctctcagccacgtcacttctgtaagtcctgccgccgttattggacacacggcggcactcttcgtgacatccccgttggtgggggtagtcgcaaaaatgccaaacgctcccgtacaatcactactaacaacaccagtagcagcactagcagctgcttgtcctccacgctctctcctcgcgactaccatcacgcgcctactccatcacacgtttctccatttttggttcctctaactgccgatcatcacggcgggccactaccctttgacgtgaagccgaatgggaacatgtgtgggagtttcacctcgttgttgagcaatactcaagggcctcatggtcttttagcactcagtgggttcgggcttggagttgggcctgcaattgaagatatgggctttggtcttggaaggcccatatggccatttcctggagtagtgtcgcataccagtgttgatcatagtaacagtaacggtgctggagctagtatgttgggcaacacgtggcagcttgctagtggagaaggtggatttgttggagctggaggagattgttttaatttcccagaacttgctatttcaacgcatggaaatggtatgaaatga

SEQ ID NO: 2 - DNA, 738 bp, Nicotiana tabacum; identical to SEQ ID NO: 1
  source 1..738: mol_type = genomic DNA

SEQ ID NO: 3 - protein, 245 aa, Nicotiana tabacum
MPSDVSERKATKQQQGAAPAPEPEHLPCPRCDSTNTKFCYYNNYNFSQPRHFCKSCRRYWTHGGTLRDIPVGGGSRKNAKRSRTITTNNTSSSTSSCLSSTLSPRDYHHAPTPSHVSPFLVPLTADHHGGPLPFDVKPNGNMCGSFTSLLSNTQGPHGLLALSGFGLGVGPAIEDMGFGLGRPIWPFPGVVSHTSVDHSNSNGAGASMLGNTWQLASGEGGFVGAGGDCFNFPELAISTHGNGMK

SEQ ID NO: 4 - DNA, 804 bp, synthetic construct
  misc_feature 1..804: note = synthetically-generated oligonucleotide
  source 1..804: mol_type = other DNA
acggatccacaagcgagaagaaaaatgccttcagacgtaagtgaacgaaaagctaccaaacaacagcaaggagctgcaccggcaccggagccggagcatcttccatgtccacgctgtgattccaccaacactaaattctgctactacaacaactacaacttctctcagccacgtcacttctgtaagtcctgccgccgttattggacacacggcggcactcttcgtgacatccccgttggtgggggtagtcgcaaaaatgccaaacgctcccgtacaatcactactaacaacaccagtagcagcactagcactgcttgtcctccacgctctctcctcgcgactaccatcacgcgcctactccatcacacgtttctccatttttggttcctctaactgccgatcatcacggcgggccactaccctttgacgtgaagccgaatgggaacatgtgtgggagtttcacctcgttgttgagcaatactcaagggcctcatggtcttttagcactcagtgggttcgggcttggagttgggcctgcaattgaagatatgggctttggtcttggaaggcccatatggccatttcctggagtagtgtcgcataccagtgttgatcatagtacagtaacggtgctggagctagtatgttgggcaacacgtggcagcttgctagtggagaaggtggatttgttggagctggaggagattgttttaatttcccagaacttgctatttcaacgcatggaaatggtatgaaatgattgtttaaggatggtatgaaatgattgtttaaggattctagagt

SEQ ID NO: 5 - DNA, 903 bp, Nicotiana tabacum
  source 1..903: mol_type = genomic DNA
atgttttctgggttctcccatttcagttgtgatgcaatcaacgcctgttgggccgctcctgtcctaatatcgttaaatattgcactgaagtgtgtaaaaaagcaatctttatctactgaagctattgaagagcggttagctggagtacctgtttatgcacttagcaatgcttctgaagaattcgtgcttgtttcgggtgttagtactggtaaaaatcttgggttattttgctttagtgaagctgatgctgaagctcttcgtcagcaaatggaatctatggaccctactatgcgaaatggctcgagggttgttcctgttgctcttaataaggttttccagctcaaagttgatggagtggcttttaggctgataccagaggcatctcaagtaaagaatgcaatgaaggaaagggaaaggactggaacgtctgatgagagcttttatggtgttccagtgtttcagtcaaggagcttaattctgaggagccagaacaagagatatcgccctgttttcttcaggaaggaggacctagagaattcactagtaagagcttctcaacagcaaggccgattgaatcctgctttgaaaggagatattcaggttgctgttctggaggatataatacaaggcatgaaggatacttcgacgtcaaagtgggacgatgttgtatttgttcctccgggtttcgatgtttcaacagatccttcccagaataactttgactatgcattaggtcctgttatgcgacaaattcttatagaaaccggagctaagatttctgaagtgtttgataattgtagacaaattctgtgtgcaattaacttctctcactcgaagtgtgacgttaatctcgatagcagtggtgatgtgtatgttattgataagctttacattttgagctga

SEQ ID NO: 6 - DNA, 8144 bp, Nicotiana tabacum
  source 1..8144: mol_type = genomic DNA
atgttttctgggttctcccatttcagttgtgatgcaatcaacgttaaattcatttaattttttgtactttataaaactaagttctcattttttttaatttcacatcctcacttgtctaatttttaaaaaaataatattcattaaatataatatatatatatatatattaaggcctcttattaagtgtttgttttaggcctgttgggccgctcctgtcctaatatcgttaaatattgcactgaagtggtaagttactcatgcatctttaattaaatattttgaattagaattaaaagaataaaattacctttgataataagcactttaccccaatagattttttaagccgtatgggtaaataaaacactcttaatataggtaaacaatttcctttttaccttgcctctctctctatcttttgttcccacccacccccacaccctcaccccctaccccccaccctctccccttttcttctagacgcaaaatctttagcagtttttaaaacctccaaaacccttttcaccattttctctctaatttcaactaaaacagaggagaaaaaaaaacatgaatatcttcaaacctaaacagtcatcttcttcttcagcaacaccacgtcaccaccacatctacagctccaacaaactttcaagaatttacaaacccatttctccaatttccttcaaaccacttttccgcaactcaacacactcaattcttctcaaaattcacaaaacccatgggctaaaattggtgttaataataataatagtgtaaaaaagcaatctttatctactgaagctattgaagagcggttagctggagtacctgtttatgcacttagcaatgcttctgaagaattcgtgcttgtttcgggtgttagtactggtaaaaatcttgggttattttgctttagtgaagctgatgctgaagctcttcgtcagcaaatggaatctatggaccctactatgcgaaatggctcgagggttgttcctgttgctcttaataaggttagcttcattaattgcttttctacaaccccattttttgtgtaatggatgtgcttattgtgttatgattaacagtagtgaagccagaaaattcaataagagtgttcaaattttgaacaacctttgtcagtgggctatgcaaggtgttcaaagtcttttttattaataacaagtaatatttgaccctatatacagaataattttaaagggtgttcagttgaccactcttggccacacatagcttcatccgtgatgattaactatgctcgacaagtagtgttatgtcgggtcaatgaagtgggatgagaaccatagcggaggcaaaagaacactaggtaatttttccttagtggatagagttatcttgtacctttgctggtgggagctagtagatacatgtagaataggtgcgcgcaagttggcacagacaccactgtcattagaaaaacttaagaaaattaaaaaatagtgttcggggggttagtacacgactacacggacttgttgtgtgtcgaatataataaatgaattgtttatgtgaggaataataatgcgaggattgttatagggtgaaaaagaatatagagattgttatgggttgagtactattgcagggatttttatgcatagattgcctacttaatatttgttcccgttgctctcgataaggttaaatttattggctgcgattggccccgacatcacagtcatctgaaaaagaaaagtaataacaaatagtgtcaaatagtgttagcggggttagcacatggactaattttgctgcgattataatgaaggatttgtttatgtggagaacaaagacaaaaatgtagggattgttatgcattgagtactagtgtggggattattatgcgtagattgcctacgtataggaatttttgtctttagatatttttatctatgtattctaatacacgtttcctattctacattctattttgcacgaaataatatatagattcctgcaaagcttacaagggtactggcaatgtggagtttaaactgccaatcaaatgctacattaatatacaaagattgtttttccttaggcagctaaacaaacattgaatttactttgtggaatttcatgttgagattatatttttatgcggcaataacgtgagcccttataaaacgtgtgtttgacttaacatgattattaagagaaacaagaattatgtcttaatcctaagttgtgattgactatataaatccccaataatcatttcgttccatttggcccgtcccatttcaattcaatgatttgggtttctctaaaccggaggttctttacattctttaagtgaaatataaatttttaaacaaactaaaaagacttgtaacaaacactaaactggtctgtagtgtactgacatgattatctcttaatcgaaaatatgggtgttttgggaatcaaagagcttaaaatttagagtaagaactgagaataagaattgtttaacatttgctgagaaatagccatgtgtttactttttataactagaacaatgacgtaaagataatatggtattggaaagaacaaaagcaagtaggacaagcatgtctaagctagtaattgtcgttgttgaatttaaatactgattatcagaggctgatagttgtattctttattgcgaaagaaaaatgttattaagtctaattgatggtcaataatgcaggttttccagctcaaagttgatggagtggcttttaggctgataccagaggcatctcaagtaaagaatgcaatgaaggtgaatatctgatgataaagatttctgtaatttttattcatgaagtcaatgttttgattttggtttgaaattctctggctgcagactaagtgtgtgcttaaatctttaagaaagctgataaagtactcatgtactcatgtcatcatgttagtatcgtgagaatttgaaattcggttgcttgtcgatcttcaagaagtgctgtctataggtcaatcagtttcttatgttcttaatcaaaaaagggcagcccggtgcactaagctcccgctatgcacccgaaaaaggccggaccacaagggtttattatacgcaacctttatcttcatttctgtaagaggctgtttccacggctcgaacccgtgacctcctggtcacatgacaacaactttactagttacaccaaggctccccttcgttcttaatcaaacaaaagattattcattttggaggtctccattagtctattatagtagagtatcatagctttccaggatcaggtattacctattctctgcttgctgagcatagtcatgatcattgcaaagtatttgcaggtattccaaagttttcgacaatctaagttctgaaatgtacttcatggccattggtcaagattttgaaagttttagtatcattatctatgaaacttttacacaatttgtatgttttaattagcatattacacactcttttattgttctagctctcccttttatgcaaccaatgtgcgagcattggttgcatacactgtcaagcataagattcttccagtttcagtacttcgtactgtcgatgattttaacatcctgtcctttgcggagtggttttcttgttcttactagtagcattagtacgccttgtcgtattgttttcagtcttaggtttctattattgtctgttgttgctttcattttggtcatcttactatcttctgttatcaatgcttcttttccatggctcctcactattgtttctggctgttttcatttcattactgttgagcttattcttcactgagccgagggtctatgggaaacaaaatctctatctccacgcttcttttccatggctcctcactattgtttctagctgttttcatttcattactgttgagcttattcttcactgagccgagggtctatgggaaacaaaatctctatctccacgagatagggtaaggtctgcgtacacactgccctccccagaccccactatgtgggattacactatgttttttgttgttgttgctgctgctgtgtgagcacttttggttattccaagattccttctctataaatatgttatgattgatgattcaacaggaatcctatctaaaacattgtaagaccccgtcttttgtttgactaaatgctaaaaacatccagagtaatgagatagcacaacaataagtagtagatacttttgcttgacgccaaatctgaaaatttgtggcacttcagattagcattaagatgtttttcttcatcttagagataaatatatagtaaacagaagttgcttttgagttgaagtggatgaaaagatagctgttgctcagtaagtttctttttacatgtaccagttgtggactccagaacatagtgtcataacatatattcggtcagtcaaatgttaggaggcctcatctttaagtgatatttctttgaggataacatttaggtttctgcttatgccaaaaatatttttgtgtatccaaatttagttcagcatgtttaaattatcattttgggcgccataactttttcctgctataatctgaagctgatcatattctgctgccattttttttaactaggaaagggaaaggactggaacgtctgatgagagcttttatggtgttccagtgtttcaggtacactctctcagctataatcttggcatcctttagcagctagacaactgccaaagcatgatataaaaagtcggtttagaaactgaaatgctcatctgccttgtcatttgtgcttactgctacgtccccgtgtgtgagtggaaacaaatgcttttttttaccttatcaaaaaaaatctttgaatgacatatgagctcaactaggatccagtaccttcaagaccggaaaagatccaatctgccttaaactgcaattaagagaatccctgtgtgcactaataaatgcccgaaatgattaaaggcaaaaagacatccgcgcaaaatattgcgaagcttcgacctctgcatctgagacagcattgcattgctctcgtttcaccttcttggatgatgttgcctggttaagaaagttcttttgtcgcttgtgtagcagcctgatgcaaatttaactttagaatcatcattaataatgtgaccactactgaggttttaagctagatatcctcttcattcatataagaaagagttctaatggcaaacctatttagtatttccttataggactcgcatgtttgtgccaatatgtttcttattttattcctaaagataccaatctcttattatgaaataagccgatctcttggatgaataagtgcgtaacatttccatgattgaatatgttgaaggatgcaaaaccatctccacaaaccgacatatgggagagttccgatgtttcacagtttgcttaaattttattattcaaaaagaatatcgtggcagacaggattcggatcctagcaaaagcaaaaaagttaggtgatttctttccatttatctaaattttggtaggcagagttatccaattcctatacttgtgagagaatgcaggtatccagtagaatatttgaggtaattttcttataaccgtgggtttaggccaacttgtgcgcaccttgactaattccacggggtacctgctatctccgggggccaccagcacaggtacggggtaactctgtctaccaaggctcgggataaacaccatgcggcagacatctgaaaatcacagtcaaataatttcacaacagatttcgtaccgttaagattcatagattgtgtgaatggtgggctgggaatttcatgccacatgacagccttttttgctatatagccacaaatatagcatactagatcttgtttttcttgtcagttctccttctcatgagtttgaaaattatagtcaaggagcttaattctgaggagccagaacaagagatatcgccctgttttcttcaggaaggttagctaactgtctggagttagtatccactaatgtgatatgatgctatatcgcccttcgtgtaaatttatcaacttgtgcaggaggacctagagaattcactagtaagagcttctcaacagcaaggccgattgaatcctgctttgaaaggagatattcaggtacgtctattgatatttacttaccgttgtgtcttagcttcaagaattatgaccgagaattttcatctgatttcacaggttgctgttctggaggatataatacaaggcatgaaggtgagaatcctcaacaagaaatttacatgttatatcgtcgagttcttgatccaacaaatgatgtcgggaaattatgcagaacttctctgtcgtcctcttactcgttttagtttcttatatatatatatttttgctaatcaagaaattgccaaagccagatagcacatggttcgaaactcagtggataatagccgcccctctacccttttccatttaaataccaagcttttgtatgcaacattgtttgaactcgtgacatgtgcctaactcacacatcatgcgttacgctgttatttatgttcatcttgcttgttaaatgcacattgccttggttaatggccgtaatggagtcggtgcgattaacaaagaatgaacatatttttgcaggatacttcgacgtcaaagtgggacgatgttgtatttgttcctccgggtttcgatgtttcaacagatccttcccagaagtaagagaacctatgtcaagtcatctcatggtgcttggccaatcctatattagcatttttgatatcatgtgtgtaaattgtagtaactttgactatgcattaggtcctgttatgcgacaaattcttatagaaaccggagctaagatttctgaagtgtttgataattgtagacaaattctgtgtgcaattaacttctctgtaggatgtcaaacggtgtggtgcggttgcagggcaaacccgcaaagatttcgccccgcaccgcatcacataatttgttgttttcaacccaccctacctgccccgcataaacccgccccccgtttaagtttttttttttcttttttttcttttttgatagtaaaaactaagattcataaacttctttttcattttttctgaaatagtattaatttaatgtaaacccaaatcctatacaagcacctccctaacaaaaccaaagtgacagtgtcttgccgttgttaccatatcaagtcttggtttatctttctttcactaattatttgcaatttaggaatacttttctaaattaatgagtttcctgtttaagttttttttttttttttttttttgattacaattgctataagcttggttgttttaacatttctttatttctgttttgcaatgagtcttttgaaagttgaaactccaatccccaaatcatcaagaaaggaaaagtacaaatagctctgagtccattttcttatttataaaggtgagaaaccatgtaatctaataagtaaaataatatatagtacaacactagtagactcttagaagcagcggcaatgcaaaagtttggatcagtatgtgtttcttggcaaaaccagcagccatacccgcgtcaacccgcacctgccctgcaaccgcaagttttttttaaaaaacaaaattccacccgccccgcttaggttcaaacccgccccgccccgtaccgctctattgccatccctacttctctgagttggcttgggccaatgtaaggaaaccatagctagtccaactatgctctacgtagaaatgactctaggtagtcattttaaagggatgttatttaaaaattaaccagtgcgtcctgaattttagtttttcagttcattttttcgggacaaaaatatcctgaattgtcctatagttaaggtatttaatttaaaatttcaagtcaaaataagtattgactaatctctaaatagcatccctgagaatgactatctgtacaattgtccctgagaatgacttggtgtaacacggatactgtggataaaatctcgagtacagatcgaaatttgttgaataatgggttactttataatattttcgcttaaatattaaacttttgtctatgatagcactcgaagtgtgacgttaatctcgatagcagtggtgatgtgtatgttattgataagctttacattttgagctga

SEQ ID NO: 7 - protein, 300 aa, Nicotiana tabacum
MFSGFSHFSCDAINACWAAPVLISLNIALKCVKKQSLSTEAIEERLAGVPVYALSNASEEFVLVSGVSTGKNLGLFCFSEADAEALRQQMESMDPTMRNGSRVVPVALNKVFQLKVDGVAFRLIPEASQVKNAMKERERTGTSDESFYGVPVFQSRSLILRSQNKRYRPVFFRKEDLENSLVRASQQQGRLNPALKGDIQVAVLEDIIQGMKDTSTSKWDDVVFVPPGFDVSTDPSQNNFDYALGPVMRQILIETGAKISEVFDNCRQILCAINFSHSKCDVNLDSSGDVYVIDKLYILS

SEQ ID NO: 8 - DNA, 881 bp, synthetic construct
  misc_feature 1..881: note = synthetically-generated oligonucleotide
  source 1..881: mol_type = other DNA
agggatccaaaaaaaacatgaatatcttcaaacctaaacagtcatcttcttcttcagcaacaccaccacatctacagctccaacaaactttcaagaatttacaaacccatttctccaatttccttcaaaccacttttccgcaactcaacacactcaattcttctcaaaattcacaaaacccatgggctaaaattggtgttaataataataatagtgtaaaaaagcaatctttatctactgaagctattgaagagcggttagctggagtacctgtttatgcacttagcaatgcttctgaagaattcgtgcttgtttcgggtgttagtactggtaaaaatcttgggttattttgctttagtgaagctgatgctgaagctcttcgtcagcaaatggaatctatggaccctactatgcgaaatggctcgagggttgttcctgttgctcttaataaggttttccagctcaaagttgatggagtggcttttaggctgataccagaggcatctcaagtaaagaatgcaatgaaggaaagggaaaggactggaacgtctgatgagagcttttatggtgttccagtgtttcagtcaaggagcttaattctgaggagccagaacaagagatatcgccctgttttcttcaggaaggaggacctagagaattcactagtaagagcttctcaacagcaaggccgattgaatcctgctttgaaaggagatattcaggttgctgttctggaggatataatacaaggcatgaaggatacttcgacgtcaaagtgggacgatgttgtatttgttcctccgggtttcgatgtttcaacagatccttcccagaagtaagagaacctatgtcaagtcatctcatggtgcttggtctagact

SEQ ID NO: 9 - DNA, 1491 bp, Nicotiana tabacum
  source 1..1491: mol_type = genomic DNA
atggttctgtccaagacagcgtcggaaagtgacgtctccatccactccactttcgcttcccgatatgttcgaacttctcttcccaggtttaagatgccagagaattctataccaaaagaagcagcatatcaaatcataaatgatgagcttatgttagatggaaatccaaggctaaatttagcatcttttgtgacaacatggatggaaccagagtgtaataagttaatgatggattccattaacaagaactacgttgacatggatgaataccctgtaaccactgagcttcagaatcgatgtgtaaatatgatagctcatttgtttaacgcaccacttggagatggagagactgcagttggagttggaactgttggatcctctgaggctattatgcttgctggattagccttcaagagaaaatggcaaaataaaatgaaagcccaagggaagccctgtgacaagcccaatattgtcactggtgccaatgtccaggtgtgttgggagaaatttgcaaggtattttgaagtggagttgaaagaagtaaaattgagtgatggatactatgtgatggaccctgagaaagctgtggaaatggtggatgagaatacaatttgtgtagctgctatcttgggttccactctcaatggtgaatttgaagatgttaagcgcttgaatgacctcttgattgagaagaacaaagaaaccgggtgggacactccaattcatgtggatgcagcaagtggtggattcattgcaccattcctttatcctgagcttgaatgggattttagattaccattggtgaagagtattaatgtgagtggtcacaaatatggtcttgtctatgctggtattggttgggccatttggaggaataaggaagacttgcctgatgaacttattttccacattaattatcttggtgctgatcaacctactttcactctcaacttctctaaaggttctagccaagtaattgctcaatattaccaacttattcgcttgggttttgagggttacaagaatgttatggagaattgtcaagaaaatgcaagggtactaagagaaggacttgaaaaaagtggaagattcaatataatctccaaagaaattggagttccattagtagctttctctcttaaagacaacagtcaacacaatgagttcgaaatttctgaaactcttagaagatttggatggattattcctgcatatactatgccaccaaatgctcaacatgtcacagttctcagagttgttattagagaagatttctcccgtacactcgcggagcgactggtgatagacattgaaaaagtcctccacgagctagacacacttccggcgagggtcaacgctaagctcgccgtggccgaggcgaatggcagcggcgtgcataagaaaacagatagagaagtgcagctggagattactgctgcatggaagaaatttgttgctgataagaagaagaagactaatggagtttgttaa

SEQ ID NO: 10 - DNA, 12936 bp, Nicotiana tabacum
  source 1..12936: mol_type = genomic DNA
atggttctgtccaagacagcgtcggaaagtgacgtctccatccactccactttcgcttcccgatatgttcgaacttctcttcccaggtacatataatatgtctcaatcttaaataagttagagttaatttacgtcgtaaattcttttattccattctgcttaaagaatttatatatttttcaaaaaaaatttgatattttttgttgtttgattgtcagaaaaagattatttgaggaatatagggaaaagtcatttttcttacttttaacttcagatcattgtgttgatcaacatttagacgttactatcaatatttaatactcataatttcaccagaatgctattaatttgcttatattattattaaccattgatctttcacccaatcaaacactaatttttttctttcagaaaataagtccattttccgagaaacattttctgaaaaataactttcgtcgtactaaacacacccatcatcgctgttgttactatgcttcttttcatttaatattgagctgaggatctttggaaatggcctctctacctttacgaggtaggagtaagatctgcgtacacactatcctcctcacaccccacttgtggaatttcactacatatgttgttgtcgtaaactaaacacacccatcatcgctgttgttactgtgcttcttttcatttttcattgagccgagagtcttcgaaaacagtctctctaccttcacgaggtaccctcgtcagatcccacctgtgagattttactggatattgactaaactcacccatcatcacttgcgctttcttttcctaagattttcttttgtataatcttgaaaatggagtttctttttgaacttttttcttttgattttttggatttttaggtttaagatgccagagaattctataccaaaagaagcagcatatcaaatcataaatgatgagcttatgttagatggaaatccaaggctaaatttagcatcttttgtgacaacatggatggaaccagagtgtaataagttaatgatggattccattaacaagaactacgttgacatggatgaataccctgtaaccactgagcttcaggcaagtctctctctcttttctggattgttgtgacggagacagaatttctgctaaaggggtaaaaaaaaaaaatttaaaagaaattgtgaccggtagaatgtatcaaggggatttaaaatataaactataatcggattttattttgtattatacagtgcaatttttttggtgaagagggttcgagttccgccccctaaattcgtccttggctgatcgtgttaaaaatattatacacggtcaagtaacttataatagaatttaacttatatacatcaatagtgtaaaaaatatatttacaattaaactttgagatcagtgatcttgaaaataaaataagtttattacaaaagataaaggtgatgtgacagtgtgtgaagaatttttagtgtttgtatatattagtttagctcaaaattataaataaatatatataaaagtaataatttgataaaaatgatactccttaatttactgatttgtaaaagatcttttatactgtcgtcaatgtgtattcttctttctcgtattctttggtaataataactgataaaatggtaattaaaacttctaagctaaattacacaaatttgtaaaagatattttacaccgaaaatatatactagtataacttgaatcttcattttcgttttctttggtaataatagtagaatgccttgttgtgacttttgcgtcacaaaaagaagaaaaatagtactgttaggtttattctggaatttattcatctctcaatgaaacttttctcaaattcaacaaagtgtgtggaacagtaaaatgtccccaaaaatatctaactttttttctacttttaagtgtagcgtaggagaataaacaatgggcaaataacaaatttaagtggtactcaagcatcataggcccacgtctttttttttttttgttccaatacatgcaataatgcaaatcatgtccttgatataagtactataaataaaatatcttgggactcccataaacttgacacaggggcgaagtcggaattttaagtttatggatttaaaatttcaatccttttaagttactgcgttctagttaataatttataaatattcaatcgattttttaagtcaaacaaaaattttggaccaaaactactgaaatcgatgcatacgctcttattccaaacacccatccaaaaaaaaagaaagacaaaaaagaaacatgccatatatataagttattagtacttcctccgtccaagttttcttgtccactataaaaaatagatatcaattgttatttatccagttcaaaaaatcaatggataatttactattttatacctattttacccttgttattaagtattagtacttatttccaattcattttcaatatataataaataggagtaatatagtaaaattattatgttatttattattttttaagacgtgtgtcgagtcaataatagacaagtaaaattggacggatagaataataaacatctattgcattttgctcatcccctaccacctgtctttgttctactttagtaaaagatctttcactttactatagcatgaaccttgctaatctttttcacggttgatagtgtaaaacaataaaagttacacctttccctcccaccaaaaatataaaataaacattgattaaaaactaagggtccaacaaaaaatataaaataaacactgattaaaactaaaggaaaaacaaacataaatgtagatccctaaatcttggaaagctttaactaaatgctccataatcttcgatctcgatactttttaatgagttttaataaatctttccgcgtctagtagcagctcctacacattttccaaatattaaaaacaaaaagggttaggtttagaaagaggaaaaagaaagatatggaccacctgagtagaaactgattcggaatttaaatttaatcgattcaatttttgaaattattaacactaaacttattatactttaaataatgtgttcaaatttaatatttattgaatattaatggttttcacatctcaatttaaattttgtgacaaaaataataaattcagttgcactcgttgctaaaataataataaaaatgacaaagatggttggacttgttcctcctttttttttttgggggcaccatagagagagttgtacttatttttctccattttgagattttttttttaattattatctcaatatttcagatagggacatcctttaaaatggacaaattgtggggtaatggaaaaataatgttgtttcctttaatttgtttttttccttatggtttactttaccgacatatacgatggcttttgccattcttctttgggtggtaatattagttttttaaaagcgagtttaaattaaatattaatagttatttttgtaggcgattgcaagtagtttaattttaattttaattttatggtacttaaaaagaccttaaaaaagaaagttgaagtttaggatttccatttttcctttaaaaaggaatattctagaagcattacagtcgacgtaaaaaaatattactcccttcggtccacaataagtgattttttggcttttattatggtccaaaataagtgatttttccagatttcaagaatgaattaattatttgtttcctacattgccgttggagtcaatagtgttggagtatggtatgagtgtttatgtgaagagatagtaaatgttaatatgatcaattttattgctaattaatattaaaaggtgaatttcttaatcagtgcgaaaacagtcgaaaaatcccttattatggaccggagagagtattaataagcaactgttttgtctaatttggcagttgatttgtttaaactagactatgattttgtgaagtttaaagtcgttttcgatctgtttgaatttgtttctcaacctagctagacgaactagtctaacaaatgtcccataattatatttaaaaaaaaaattccagaaagtctgattttttgttattaaagaaaagtcaattagtgactcctggccccaccacgcaaagagttcaaatttctgaaaatttgattagtttagttaatgcataaattaattaatgcataaattttgacgggggatttaaactaaagtgaatgaatgaccatatgccacaattttttaagcagatattttttcctattaccattttatccttcactctgtgctattactccctctatctcatattatgtgtcgtgtttttttattacacgcctcttaagaaatattaattaggaaagggattggactattctacccttatttatgtcttaagatataatttatcttcattgaatatttattctatttatattttatcttcatcttcaagaacaattattactaagggtaaaaaaggaaaaaaatatcaattttgtcttgaacttctaaaatgataaataatttgagacaattatttttagtaaccatgactgttaatatgagacgaagggtgtatagcataggactttctaccacctgtgatcttatatcccctttacatattatgtaaactcataaattgtttaataacattattattttaatttaagttatagcccctttacatattattttattatgcatttttatggtactaatatatcggctcctgttgctttttttgagccgaaggtctcctggaaacagcctctctacccttcggggtaggggtaaggtctgcgtacatattaccctcctcataccccacctgtgagattataccgggttgttgttgttgttgttatatatacatcaatactgaacaaaaattttacaccttcaggccagatttatctattgtagcaagtatattgacttatttttatattgcaaatttcacactaaaaggaagcttacctacaaatattctttgggagtatgagtgacgtgataatgaaaaattttcacagacgatgtatataacttaaaatcaaaaattatatcgacaaactaagaatacgtttaatttgtcagaattaaataattttaaatctttttatttttttattttattttgcagaatcgatgtgtaaatatgatagctcatttgtttaacgcaccacttggagatggagagactgcagttggagttggaactgttggatcctctgaggctattatgcttgctggattagccttcaagagaaaatggcaaaataaaatgaaagcccaagggaagccctgtgacaagcccaatattgtcactggtgccaatgtccaggtaagataaatctatatttttagccgctcctaaaaattaataaccgacgaaatatatatttgttggtatgcatgtattatatacatatctagtaaatgcaattagtttcagccgatcggttaattttgtattttggccaatgttagcctatcatggatctttacacaaatagccgattgaattcattatttaattttgaaatttttacacaaataagcggatatattaactatttactttttctatgtatatatacattgattatatataattatacacatataatgttgttccttctgtatcctctacttgtaaataatgattttggaaattagaacgttagcttataaacgtaaatataaataaaacagaaattaattcgagcccactgaattcacagtgtttccttaaggaatttagtcccctcctagtacccaaggttatggattatttcctcccaggatagaacgaattacacactggtgtagcggtacttcaaaccccagtgtttcagcgaacacaaagttccgcagcaaatcacacttatggatgctttgtttgtagttaaaaataatgcagaataaggaggacagtcgaatggaaattctgagaggaaggaatgcaaagtatagccaatgttgaattcttactgaagagaatatcagattgcaattcgtttttccagtgtatacgcagtgtatacagagtgtatatccagtatatacagagtgtatatttagtgtatacagagtgttttgagtgttttttccgatgtgttcttctttctctccaacttatgctctatttatagcagttatttgagagaaatccgcccctccatggtgcaaaggtgtttggcaaaggtgcaagttcctaaacccaaaggaccgaaaacagttgattgtgttttcataggatatgcgactaatagtaaagcatatcgatttctggttcataaatcagaaaatcccgacattcataataatacggttatagaatcagataatgctgagttctttgaaaatatatatccgtataaaaaggaatgtgagtcgattggtgaaggatctaaacgacttcgggaagaaacaaaagaaagtacatttaatcagggggatccaagacgtagtaaacgtcaaagaacgtctacttcgtttggaccagattgtggtgcaaattggatcaccggttcaactgattcgaagtccacaagtggatatgtattcactattggtggaggagcggtatcttggaagtcgtccaaacaaacatgtattgcccgctctacaatggaggctgagttcatagccttaaataaagccggtgaagaagcttaatggctccggaatttcttggaagacattccattttggcctaaatcgttggcaccaatatgcatacattatgatagccaaacaacaattggaagggctgggagcgttatgtataatggtaaatctcgtcatatacggcgaagacataaaaccgttaggcaacttttctctagaggaattatcacaattgactatgtaaagtcaagtgataatgtgtcggatccacttacaaaaggcctaactagagaggtagttgagaaatcatcgaggggaatgggactatggccgagaacaagtcattgtggcggtaactctacctagaagactggagatcccaagatctaggttcaaggagatcaaacaaagtcattaatgacggttcaacattgtcaacaaatatttttagtccattctcgtgatgagacaatgttcagtaccaaggataaaacattaaggctttttaatgatttctaaatttaatacaaggtatatcaaatagtgtatctaagggatgacacgtttaggaatcacctatgtaagtgtgaagtattagccgcttcaatgagaattttgcaaggccaattccctacgcacttatgaaaccaggcggtgttcatggctgaaacgaacacaacaatgagaaccaaagacggttaagggattgattgtgtgacttatggttgtctaggtatacactaaagttcgacggttcaaagatatcaaatctaccgattgaccgagtatatccgacataagttcactacggaaagttcaaagggaaacctacttatccagatgcaattaatccttgtttgcaaattacacaagtttttcatgcatacttccgtgatatagccattccccattcatgtgggggattgttgagtttttgtttaagatgaaatagtcttaaaattagagtgaatggaaaatgggatttggatttggatttggattcggatttggtcaaatgatcgatcgattgattaattttttggaccaaatttatttgttaatagtgaatattaacgtgatataatccgtgtttgtaacggatgttttccaatccgtgtattgtattgcaacactaagcaataagcagcctagtgcacctcccacaatggtgcaagtgctcctcccaccaagcaagtgtacattccaccatgtaagtatttactccatgatagcctactgcttgttgcaccatggagggggaggatttctctcaaataactactataaatagagcataagttggagagaaagaagaacatatcggaaaaaaacactcgaaacactctgtatatacttaatatacactctgtatacactggatattcactctgtatatattggatatacactctgtatacactgcgtatacactggaaaaataaattgcaatctgatattctttttagtaagaattcaacattggctatactttgcattcctttctctcagaattttcattaaattccttaagcttgttgttccttctgtatcctgtacttgtaaataatgattctggaaattagaacgttagcttataaacgtaaatataaataaaacacaaattaattcgagcccactgaattcatagtgtttccttaaggaatttaatcccctcctagtacccaaggttatggattatttcctcccgaattacacactggtgtagcggtacttcaaaccccagtgtttcagcgaacacaaagttccgcagcaaatcacacttatggatgctttgtttgtagttaaaaataatgcagaataaggaggacaactcagaagtcgaatggaaattctgagaggaaggaatacaaagtatagccaatgttgaattcttactgaagagaatatcagattgcaattcgtttttccagtgtatacgcagtgtatatgaagtgtatatccagtatatacagagtgtatatccagtgtatatagagtgtatattaagtgtatacagagtgtttcgagtgttttttccaatgtgttcttctttctctccaacttatgttctatttatagcagttatttgagagaaatacgccccctccatggtgaaacaagcactaggctatcatggaggaagcacttacatggtggaatgtacacttgcttggtgggaggagcacttgcaccattgtgggaggtgcactaggctgcttattgcttagtgttgcagcacaatacacggattggaaaacatccgttacaaacacggattatatcacgttaatattcactattaacaaataaatttggtccaaaaaattatcaatcgatcgatcatttgacaaaatccaaatccaaattccatttctcattcactctaattttaaaactatttcatcttaaacaaaaactcaacataataatacataaattatgcatgtattatctattcagcggttattgttaatttaaatggtagggtggtcggctatttgggctaattcttctttacttttcttagctgatatacatagattatacattaattatacaaatttttacatccgatgcaatcagtttaaacggctaggtggatggctatttagattaaaactcctctaattttattaaatataaatcatttttgttaccataagtgtatttaatatgttgtgatagataatatatcttactttctaggctactaattcaattttttatggactgtcacctgtatttatcttttatagcctgtttggccaagtttctttttggccaaaagttttttttttttttttgccaaaagcacttttagccaaaaattgaggtgtttggccaagcttttagaaggaaaaaagtgtttttgaggagaagcataagcagttttggagaagcagaaaaaagtatcttctctccaaaagcacttttttgagaagcactttggagaaaaatacatttagaagcagttttttaaagctgggtcaaacactaattgctgctcaaaagtgcttttcaaactaattagccaaacacaaactgcttctcaccaaaattacgtttgagaaaagcacttttgaaaaaaacacttctcaaaataagctgatttttgcagtttggccaaacgtttaaatttttttgcactatcgtgtagagaagttaaactctataaaacacatggatttgtttccaaaatttaggaattgagagtgctaattttgtaattttaacaggtgtgttgggagaaatttgcaaggtattttgaagtggagttgaaagaagtaaaattgagtgatggatactatgtgatggaccctgagaaagctgtggaaatggtggatgagaatacaatttgtgtagctgctatcttgggttccactctcaatggtgaatttgaagatgttaagcgcttgaatgacctcttgattgagaagaacaaagaaaccgggtaagtcacgatttcgtagagctttttggttgttaagttatagtgtcgaccgaataatatccaataattccgacctttctggtcaatggtgaatacgtgtttgttcaaatatctttttttttttccttctctttgtatgaatatcacaatagaaggggagtcttggagcaacagtaaagttatctccgtgtgatctacagatcacgggttcgagctatgaaaacaaccactaatgtctgtattaggataggttgtctacatcacaccctttgtggtgcgattcttccccgaaccctgcgtgaatacggaatactttatgcaccgagctgcttttttatatatgtaaatatcataatatatagttgataatttgtaacaggtgggacactccaattcatgtggatgcagcaagtggtggattcattgcaccattcctttatcctgagcttgaatgggattttagattaccattggtgaagagtattaatgtgagtggtcacaaatatggtcttgtctatgctggtattggttgggccatttggaggaataaggaagacttgcctgatgaacttattttccacattaattatcttggtgctgatcaacctactttcactctcaacttctctaaaggtataattccttcattacaagtccttaaatatgtgcttaatccaaggcgaactaggatttgaggttaatgagttatggacttgtcatcaaatccataactcgtcaaaatcgagttcggatgatgccattctctttttctgaaggggaactttagagcaacgataaagttgtctctatgtgacttataggtcacgggtttgggccatgtaagatgccgctaatgcttgcattaaggtcagttgtttatattacattccttggggtatggtccttccgcaaaccctatgtgaatgcatgatgctttatgcaccaagctgccttttactatcataaaacatatgatgctctaattagctagatatatatatttctaaggctaaacattgaaaagtaccactagagtcatatcactagccttacaaatctaatcatttttgctattgtaatgcaggttctagccaagtaattgctcaatattaccaacttattcgcttgggttttgaggtaagttaagattaattttgttattttcaatggagctatgggcaggcaattgctatcattacttctaagttctattagtacaatttaatcaactatcaattgcataaaaatacgagtttaaattatatacatcatcattctaactaatttttacactatcatattacttttatgtgttatatcaagtaaccagtcttaatttaaagattacaaatctcgcattttatggtgtctagcctatggatatcttttgaatgacctgatgtaagaaacttgaactctaaaaataatatctttttcacaaacaacacactttaaatatactctttacaatctgaaatttccgaaagtgaagcctcggcgtaactggtaatgttgctacaatgtgaccagaaggtcacgggttcgatccgtaaaaacagactcttgtagaaatacagggtaaggctgcctacaataggctcttgtggtccggccctttccccgaccccacgcatatcgagagcttagtacattagatttttttttcttcacaatctgaaacttctatgttaaaacagggttacaagaatgttatggagaattgtcaagaaaatgcaagggtactaagagaaggacttgaaaaaagtggaagattcaatataatctccaaagaaattggagttccattagtagctttctctcttaaagacaacagtcaacacaatgagttcgaaatttctgaaactcttagaagatttggatggattattcctgcatatactatgccaccaaatgctcaacatgtcacagttctcagagttgttattagagaagatttctcccgtacactcgcggagcgactggtaagtatcgttacgcctcagttccaaataagttgaaattagactatataaatcgttacttcttcatttaaacgaaaagttgtgtcttacaatgaaaggacatttcaacccatgcaaaagttttaaaaaaaaagatattcttgtcttggcatctattatcaaaatatcttttttcttttcctccaaaaaacacttcttttctttccaaattatgtacttaaaaaaaaacttgaaatatgtcatgctttactaaccaaaactctccactatttcattagcacagaaatttgaaagacttgtcacttgttatatttactagaagaattaaccctaatagcagctcagtcgaccacttaaataaaaaataatcagtagatgaataatatatgcataattaatatacaaaggctatttgcattcaatccctttgtgtttttttaatcgtactttaaattttagtgggcgtttggacttaagaattgtaaaattccattaaaaaaaagtgaactttttttgaagtgaaaatgatttttaaaaattagagttgtgtttagatattaatataatttcggttgtttttgaattttttgtgagtgatccgagtaaaaaattttaaaaacagcctgttggagtttttcaaattttcgaaaaattctaaaatttatttttccgattcaacaggtgatagacattgaaaaagtcctccacgagctagacacacttccggcgagggtcaacgctaagctcgccgtggccgaggcgaatggcagcggcgtgcataagaaaacagatagagaagtgcagctggagattactgctgcatggaagaaatttgttgctgataagaagaagaagactaatggagtttgttaa

SEQ ID NO: 11 - protein, 496 aa, Nicotiana tabacum
MVLSKTASESDVSIHSTFASRYVRTSLPRFKMPENSIPKEAAYQIINDELMLDGNPRLNLASFVTTWMEPECNKLMMDSINKNYVDMDEYPVTTELQNRCVNMIAHLFNAPLGDGETAVGVGTVGSSEAIMLAGLAFKRKWQNKMKAQGKPCDKPNIVTGANVQVCWEKFARYFEVELKEVKLSDGYYVMDPEKAVEMVDENTICVAAILGSTLNGEFEDVKRLNDLLIEKNKETGWDTPIHVDAASGGFIAPFLYPELEWDFRLPLVKSINVSGHKYGLVYAGIGWAIWRNKEDLPDELIFHINYLGADQPTFTLNFSKGSSQVIAQYYQLIRLGFEGYKNVMENCQENARVLREGLEKSGRFNIISKEIGVPLVAFSLKDNSQHNEFEISETLRRFGWIIPAYTMPPNAQHVTVLRVVIREDFSRTLAERLVIDIEKVLHELDTLPARVNAKLAVAEANGSGVHKKTDREVQLEITAAWKKFVADKKKKTNGVC

SEQ ID NO: 12 - DNA, 526 bp, synthetic construct
  misc_feature 1..526: note = synthetically-generated oligonucleotide
  source 1..526: mol_type = other DNA
aattggatccgagttgaaagaagtaaaattgagtgatggatactatgtgatggaccctgagaaagctgtggaaatggtggatgagaataccatttgtgttgctgctatcttaggttcaacactcaatggtgaatttgaagatgttaagcgtttgaatgaccttttgattgagaagaacaaagaaacctaataagatcttcaacacctacaccatttttttaatcactactacccattgcattgaacaaacttccaagttcttcttagcttcagattaagaaagtaccctttcttggctttgttgatgtggtaccattgtccattgtcttgtgtgtttccggtttctttgttcttctcaatcaaaaggtcattcaaacgcttaacatcttcaaattcaccattgagtgttgaacctaagatagcagcaacacaaatggtattctcatccaccatttccacagctttctcagggtccatcacatagtatccatcactcaattttacttctttcaactctctagaaatt

SEQ ID NO: 13 - DNA, 708 bp, Nicotiana tabacum
  source 1..708: mol_type = genomic DNA
atggggagaggaaaaattgtgattcaaaggattgacaatacaacaagcaggcaagtgactttctcaaagagaagaaatgggctgctgaagaaggctaaagagctagcaattctttgtgatgctgaagttggactcattattttctccagcactggaaaactctatgaattttccagcaacagcatgagggctataattgatagatacaacgagatgaaggaggagcatcataacctcatgagtcctatgtcagaggtcaagttttggcaaagggaaatagcaagtttgaggcaacaactacattacttgcaagaaaatcacaggcagcttctgggtgaagaactttctggactcagcatcaaagatctaacaaatcttgaaaaccaacttgaaatgagtttgaaaggcgttcgaaagcaaaaggagcagattttgactgatgaaatcagagaactaaatcagaagggcaatcttattcatcaagaaaatatagaactctataaaaaggtaaacctcattcgacaagagaatacagaattgaagaaaaaggtttctgagcaaggatgtggaagtgaaccaaatgggggtgttcaggccacacatgcaatcagcaaaggatatgatgtacatgcaccaatcagtctccagctaagccagccacaaacacagaagagtgtaacatcaacaagcgtcatgcagcttgggtaa

SEQ ID NO: 14 - DNA, 15537 bp, Nicotiana tabacum
  source 1..15537: mol_type = genomic DNA
atggggagaggaaaaattgtgattcaaaggattgacaatacaacaagcaggcaagtgactttctcaaagagaagaaatgggctgctgaagaaggctaaagagctagcaattctttgtgatgctgaagttggactcattattttctccagcactggaaaactctatgaattttccagcaacaggttagttatttttacatttttaattttaattttaatttggtcctaaacatagagatatataatttttgcattcttccttttttttttcctttttctttgggaaaagtttggactgaaatatgggattttttcctgctgtagaactgcctctatttcatatattctgtacttcttgcatatcgttttatgtaatttttttgtttaaaaattaccaatttggtacctttccatttactgctattttttattaaaaattattaccaagttggtatctttccagtttgtactttgtatgtgaaaaaagagtagtcagatcatatcatggattaacctttgttactgcatcaagattccaacgtgcatatttataaatgcatgctacgttactttcagttgttttttcttttcttttttttttccaagtatattgggagaaatctaaaactagctttgcttttttaatccagggattaaatttgttaatcatgcacgaggtgttttatctagcctggcccattctatacaaacctaattaatgttgaaaaaatgactcgaggaacaacatgcaccaatcctaaagaaaacctaataggagtagtaatatttgcaaatctgaaatctgtccgcactttagaatttggtttaaagccctgcagtgataaatatctctattgtattctatttggtgcaagtaagaaataactctttagagcacgtttggatgggaaacatgttataccatcattaattattccggaattagttatcccgagattagttatttacccttacaatagggataaacaaacactacaaatctacaaccccgggataattatcccaggattaattatcccatatccctcgatattgttattctctcttcagtcttcacataaaccctaaggtgtcatttggtttaatgattagaataaaggcatatgagatttaatacaattcgtgtccttgatcttattgagttaatcccacatcagtgaatgcaaacattattatgctgccaaatatattattctactaaactaacaaacaggatgctccttaattctacgtatgcatccagaaacttctgtcactcttttgtggccctcagataaactcacataagtagcttctcacataattagaatactttgtactacctttctatacaaccttctttagacgcatgcataatttacattagaacttttaatcttttcatcaagatatacttgtgatcactttaattatgtgtatattttgcttcgaaattgcctaaatagaatttcgtcttatggaatatagactcacaagtgcagaatcatacgtatagtttaatatggggctcagataactagtttaacacatgaatttgactcatacactaacaatgtaacaatttataagctgttagtgtattttggcgagttacctgtcttattactcctacttattatgcatcatggataattacctataattatcttttaggtgatttgataatgtaaagtttaaattcattatgttagtgtgagaagttaaactcttgacacaattatatatttgctatgttaccagcatgagggctataattgatagatacaacgagatgaaggaggagcatcataacctcatgagtcctatgtcagaggtcaaggtatagctctttaattatatttaaatctgcttcagaattgcctaaatagaattcgtttgatggaataatggacttgcaaaggtagaatcatatgtatagtttaatttggcacacagataactagtttaacacaggaatttaactcatacatatacattggttatatacataaacaccaccgatttttgttttcacaagtggttgttaatagagaattgcgagtgtatgtaatgtccctttttggatgttcttgtatactagactagatgtgttacatcgtactcaataccttgtgagaagggatttaggaaaagtctgctctcattctatgtcagagaatctaaacttccaattaaaaatctttaagataatggattaaatattctttgctatttaaccttttaaatgatcttgtatatccattgtgggatatatatatattgagtaactcatcaaatgtggagcagataggctcgaactaaatgttatagttggaaattttcaggttattattcttatccttcaaccacgcgtgtggacctctatgggaaacattatctatttaatttttcattttggttagctcaaatatattctgtcgaaatctcaatcaaagatagtataaggccagaatttgaaagatgattcattttttaaagttccacgtgatatctttataagctaaatatgaatgatgcaagctttcagtcaaagagattgaaacagtcattaatcaactatggcacccctaagattgacctcttttttaaatgaacctcttaccctggcctcaacaatagcttttgagaaaattgattgctaaatgatttcatattactttatgttacaattaattgacaagtactttgattaaacagtaaatcggcgaaggatcagattagccacaatgacatcgacaatttttttcttaacagtgctgtaacagagaagcccagaaagagcacttatagtctccattatagaacaagagctacaccattctgtttgacaatagttgatgatggcaagttagctgtagcttcatttcccttttgaggtaggaattgtataaaaggcttggaattgtaaaaatacaaaaatttaaaaatgcaaattaatggagagttgactaactcccaaaaatagattgctatggagagagaacagatgtgaataatagctcttgatcttcacacatggtcaaggtgcgactagattaaagcaaatacaaattcttcaagaacaacaaaaggtttttaagcaggttttttcaagcaagctcagttgaaggctcacagtggagtttagggagggtcaaaaaagttttcatgatgatcaaggtcatcctttggcccaaactaactccatattatgttatcagccttttcccattcctttattctcgagttcaccccgtctttatcatggctcacggaagtcttgcaaaaagtttcaagaccgcaggatccacctatgtgaatttcgtgtatgcattaagaattcagtaacagtaggcattcatctgaagtggctgctctttctgggcatcttgccccattcaaacttttagctatgttagcgtggtcagtgttcttcctctggcgccaaggatggctaggaaataccaatggaacacctgaactgggacaatccacccaccaggttttggttggtatatgtcagattcgttaactgactccatggtaacttctggtaggtatcttctcaacatggttatgagagagattcaacttactagaaaaacagaacacgatattagagagccaaacagtcagaaaagttaatgacatatagcaaaagctaaaacacgaaatgtaaatcagcaaactcatagagaaaggataaacagaatctttgctgtgcaccttagtgactgggtgaggagggggaataatcctcactttgttgatgttctggttgtttttgctgcctttactgcttcatttttcctctctgcctgctggggaatttcatcaacttttttttttattaaaaaaagctacccattataagacttcacacattcttgtggtgcaatgctacacttctggaaacttccatgtgttggttggtactcttttatctttttccacttccatactcttcttgtacatcctctcatgtatcttcttccatggttctgctatcttcttgttattctcctttgccctcttattcctgcttattaattgttggctgattccctgaatagatgcctatcttgttgtcccttttttccttctccccctacttcatgttcggttcttatgcttgctcgagacaacaacctgcttcctcctaattggtcacttcaagcatcgatagttaccattgactgttaaccttcttccaatgcaaatatatctgactcattagttctatctatgatgtgttgtcaattgatgttgatgagatggtttagtaaacataaagggggatggagtccctgttggaggtgtttgtgttgaaaggcctctagtatcattccctcgcgctataatccctctccctgttccttaatctaggaagctcgataaaccttgggattaactccagtcaaatacttgattcctgttctttccttggagcagttcgatcaagtggaatcaaatcctacaggctcatatgctaacactgccactcagttactctataagatttctataagcatgaagagaattttgtgtcaaatgctgtatctcatgacgatgtaaagaaagttaaatggtaagcttattttgaaaaggattttggaatcgtgctttgatcccaaaaggtgctgccttcctctattagggttaaagctttattagcacaaattgggacttctgttagggtttgttccggtgccaattaattcgtaattgtggcctattgtaacctaactacacgctcattcagagttccaaacaagttttactttatcaaatccaaggatcgattcttgaattgcaacacctacgctattatatgtcatactagtgaactactttgatggaacctaaacaaatacagtaagtcatctataatcatctataagatactgaaaacagaaacccaacctaaaataactaacaaggtctcttctcaaagttgagtgatccatagttacctccatgtttccactactaacctgaaaaatccatgagctgtgtagagaattcccttaaagcctccactacactctggttcctgtattgtctgcattacaaaatagcaggtttgaacctgtttcaggaagtttttcctaccctccttcccaatagtaagcatagtcccataagtgaaaggaagcaataagtatcaactcttaatcaagtggtaaatactatcatcttaagagttatccagcatgatagatacttattatccatctacataatataccacttatcatatttgaatcaggtcatcatctccagagacagttatagattctggctgatgcagcatttgttgttccaacaagttctatataagcagaaagccaaaccacaagaaataccatatcagatcaaacagatgcatgttggaaattggaatagtttatcccagacaaatagtgcaggaacaaactgataccggttctttttcccagtttatgttgcactagttttgagaccaaaagtcagccatcaaacagtcatgatgcctgaaataagggaggtcaccggagaataaggtggcgacaaaactagtaatgctcacccgttacagtttaattccactccaccatgtgcactgacaagagaatataacctaggggaatctgcctgcagatgagataattaggctaacaagcagaaggaatacgatggtcttgacatgataagccggagtttagagtttgtttatctctgcataagcagaatccatgatctaattaatcaactaaaccataatccaaaatcagttgaggccggctatatgttcctatatatccattctgctctgaaactgggatctaatgcctctttaatttgcatttgcagaaatgaagtgaaatcccaaaaagacatctgcaatgctaggttgaaggtccagatgagacactattttaacaaattccattagaggatagttcgtttgtctaacagagaatgctttaaaattgcagatccctatatcagaggagaggatggaggatgtctcaaccccacctaaatcgatataggatctactctcagaggtaagtaatataaaattttcaaaaaggaagtcattccacaaacacaaaatataaaaactcagttggatgaaatgtctacacaagtttagcgattgaaaaccctaccaaaggtcctacgtgacacattatgacaatttgctcaacatcgcaactatttatagattaaattagatggaaaacctaagtttgaattcctatccatggataccgtaattaaattataaagataaatagtcttccccatctagggagcatagttcatggagcaaattcatgtgataattcaacaaatggaacctaacatatctaataaataacctgaacctgagtgtcctggtcttctgtctcaaactagcatctattcctggtattgcatctcaaatatgaccaaatccttcactggtgctaaatcctacttttgtgctctcaagttagcatctattcccttcgttagtttacccactgtaactgacggtcacgtccaacgagagcgtgagcatcacactattctttcttctcatggaggcattctactagcaaagaagactcctataagtggtagcaccttgttggggctttgtggtataattctttgccgaaagaggtctctccagtgttgcatgtttatatctacgaccgatccctcaccagagctgacggacatctcaggtaaccatccttgacttaccgacatgtcaatgaggatagcaaaaaaggtatccaactgggaagaaggcagtgagggtataagtattatcagaaatttcaaatgacaagaaagtaaaagttcaggtgctccattaaatccaaatcatatatgtcaatgaccaagggcatgattaatcaaaacataccatggaatatggattcatggttttctaaggctggaggcaagcaaggacattaggaaaatcttgcaatgattcctgaagcagtcattccggttggaaaaaaacacccttgaggccttgaccatccgaggaggataacctgagagtaaatggacatgaacacagatgaaaatattcacatacatatcaagttcataatccatccacaggagttctctatattcctccaggacttcaatctcactacgaatccgtcattaaattgtcaatggtaaaagtttaaaaattgcatccttccagcctgaaagacaatatcaaccccaaccaatgtatgtgctatcttcagacgttcatcgctaaagagtttgtttcttattgctatggaaaaactgagacacgatacatttttgccgtacattgtttaccactagttgatctctattatcaaacacgggtgtcaagttccattttacctgttcaaaaaaagggtgtcaagtttcctatttagaaaattgggcagggagcctacaggtcgaagagatgcacaattacaatctcagagatccaactgtctaaagagttgacgcacaataggggtgtacaaatgaaaccgacaaaccgcaccaaccagataatccgagtcaaaccggaaaaaaaaaccgcctatagtttggtttgatttggtttggtgttggaaaaaaaaatccgaccatatttggtttagttttaactaaaaaaaagtcaaagcgaaaccaaaccaacccgacattatatgtgtagaagttttaaatatatttaatgcataaaaatatttattgtagtgtagtttataaatatttcttaaattttttcataattttatcttttaacgtattatttcaaacttgacttataatttttggatgctccaataaattttatagtccataaatgttagtaactcaaataaatcctaaaccaaaatcaaatcaatactaatgctaataaaaaacattcaattcaattgtactatgaatgaaaatagtgttggatatctatttttaagcttattcatgatttagataaaatgcataacttatttttcttttatagtgtttagtcatgtaaataatagtatatagtcgtaatttttaaattatgtttgttttcattatggcttattaataatatttattttgtgcaattttattatctttattattgaatattttagtacaatgccataactcatccaatatttatgttattttattgaaaaacaccttatatagttctgtcttactagattaaagaaatatttggagcaaaaattctatgttttgtgctatgaagactttatgaaaaaaatccggaaaacccgaaaatccgaaaaatccgagaaaaaacgagactaaaaaacctgacttttattggtttggtttggtttttagatttaacaatccgatacaattggtttggtttggtaattagaaaatccgaaccaactcgacctatgtacacccctaacgcacaacaatgcagatggagttcggggtgcttatcgggtggataaaattacgcttaacggttatcggattataaatgttgtaatccgctaaccatccaataagataatgggcagattggtatcgaattgacaattatcgggcagtttatcggctaaacaaaataaaaaacttttgaacaatcaagtacatacaagaactactgataaggaagacataacagatacgagctaatctatgctaaacatgaagaaaactagcttcctccatcttactacagatctacctaactttattgagctataatatgagccaacggtagaagttagggatttagagcctgtttggatgagctgatttgatgtacctgataagcattaggtgctgaaaaacacttttaagtcttgaaactgatttaataaataagcagttacgcgtttggatacaagtgctgaaattgataataagctgctacagtatttgataaaaaagtgctgataagctttttttctgttaaaatgacttaaataaccttataagtatttacacctataagggcgtaatatcttcagaattttagattccaaatcgatccaaatacaaaatattctatttgtcattttattttaaatacaactgtgcttcgataagataattttatgataaatataatttattttatgacaagcatataatcataagttataataattaataaattgataaaagtttctcagtaaaaaataaacctaaataggacaaaatatttgaagagaaacaaacactgtctttatcagcacaacacgtagaaagcaacataccaaaaatttgatatgtcctcatttattatatacaattcaaagattaatacacaaatcacatagatataaatatgcaaaggaatagagaatttgcttatcttaaatagtcaatgagcattgcagacttgaagaggcggagggagggggttaggttgaaatagtactcgaggcattgagtatcgatgcaaataacattatacgtgtttacacctataagggcgtaatatcttcaaaattttagattccaaatcgatccaaatacaaaatactctatttgtcattttattttaaatacaactatgcttagataagataacttttatgataaatataatttattttatgataagcatataatcataagttataataattaataaattgataaaagtttcttagtaaaaaataaacctaaataggacaaaatatttgaagagaaacaaacactgtctttatcagcacaacacttagaaagcaacataccaaaaatttgatatgtcctcatttattacatacaattcaaagattaatatacaaaccatatagatataaatatgcaaaggaatagagaatttgcttatcttaaatagtcaatgagcgttgcagacttgaagaggcggagggaggggtttaggttgaaatagtacttgaggcattgagtatcgatgcaagagttttgttctaaaaaagaatattttaaagataaaataataaaaattttggtcaaacttaaagtgcttataagctaaaaatttataagttgggggttactagcttacggcttattttttacttataagcacttggcttataagtacttttaactttaccaaacgcgtagataagctactttgaccagcttataagcttagccaaacaccctcttagccgtttagggtttcaatagtactttatatacataggggaaaaaatgtaaatataaaaattcttaacgggttaacggtttacccgataagaaatttgagtaatctgccccaaatcgttaagccgttaattataaaatctcaatctgttcaccatccattaccccgataatccgataccaataagccaataagccatcggtttggttggttaacggttttggtttgattttgaacagccctagatggtgtttttggctaagagattatttctacaaccaagctctgcattcgtaataaatgtttttttctccctgaagaccagggtccgcttggtacatggattgcgcatatgataggatatgtaacttcgtatgcatgtattctcgacaagattaattaactcaatctggtgaggacaagattattaattaggcaggtactcaatagagaatttgcttattacattctatataattcattgctcattattccacatagtcattcctagggcattaaacttcatagttacccgtgataatcacaggtccccaatagatgattagttgaccaaagatcccaaatgtctaaggtgctgaaagaccaagaatgcatatgtaaattttgtgagaagttaattcttaaaccatgttgagcaattgtaacagaggtttctttcttgcttcctgaagattagctgtcatttcttacacagactgctcacctgaaaaggtttacctcccccagtttttagctaggtacattaatcttaatcttaagagactagaaaactgatcggcagataactattaatctaggccagcaattagtataagaggcaaaaggttcaccatacaaatagaggataagcccttctttcatttcttttcctcaatctcctcctcttaatcctttcttccaggtccaacttctgctttccccaacccacccacccacacgtcagtccttgaccgtccacatgcacctttttccccccaacctttcccactatgcaatttcaaccctcaccccttgggaactaggggtgggcattggtcggttcggtttggttttgtagaagttcggtttagtttattcggttttcggtttgtgattttaataaccaaaaccaaatcataataacttcggtttggttcggtttattcatgttcgattcggtttttcggtttcaagccatggcaaaaatagtatgaaacaacgaaaatagattacttgtgagagaatagccaaaaagggtaaccaaaaacatattgagtgcttataaatattctccatttatttttgttagactgcattaatggtcatataatataatgaaattctatatatatccaatcagtgaaataagtgaatgattagcatgcgaattcagatgatacggaagaatgaattgtgcgaatgaggctttgtttatttttctgtagatcccattcacccgttactaaaattcaaatacgaagaaggattacatcaattccgcaatatccgttggggttaaaattcttgttgtattttatattgttcttaattcggttataaataattcggttttgggtttaggctaaaaaaattcggtttttcggtttaaccgaaaaccgaaccattaaaaccgaccaccgaactttttaaaactataaaccgcaaaccgaccgaataaaccgaaaaatcgaaaccgaataaaccgaaattttcggttcggccgattttttcggttcggttcggtcggtattatgcccacccctattaggaacccaattatttttgcctctccttaacatgatccttcttctttccttccatgacatcattccctcctccctccacccatccctaacattttacaaactatcgtggcattaaaagtatgaagcatgctatgaaattttggaaaagagcgatagatcataaacttacggacaccataaaagtaatgtagaaccattttggggtagatatacaacgaaggctatatgctaagaagattcatgaaaatttattgcaagaggaagaatctacacagcttagagaaagcgaatgagaagagtgccaagaaaagcttttggtgggtaatgaagaagaaaggaactcatattaaatatataatatcataaatgatatatgaaatagttgtcactagtgtgagaacagtagtacgagatacaaagagtttcccataaccatgtttttacacccagaataggcattgagccttgtttaccctacttatggatgagctaactcacactatatagtttgaggttacattgtacatgctattttcaaatggcattgtgctaattgacgaaaccatcaaggatatcaaccaaaacttgaacttctgatacgcactctaaaaagttgaagttttaggataagtaaaagtaagacatgcattacaagtttagcccgcataagaagagtgaagttgaggtcagaccaaacaagattgcaaggtttaaaagcaaccaattcatatatctaggcttgatgttcgaagagaacggcacaatagatggggatgttacacatccaatcaaaatagagtggtttaaatggacatttgctaccggagtattatgtaatagagggataactaccaaagtgaaatgcaaattctataaaacgattgtaagaccagcaatattacatacatgggagtaaatgtaggtctctaaaggctccaagtatccacaagataagtggcgcaaagatgaatgatcaagatccaaattcagatgaatgtatttactttctataaactttcaagttacgaaaaagtgctacatagataggtttgaacaaatgagaatagatatcgaggattcatatagttgagcccgtctagttttagattgaatcgtagttgaaacaaaactaagaacgttctacatctaatatgtggctctcatcatcctaaagtacttttcttgtatggttgaatttatcatgggttgcaataatcatcaagcactacaaagaaagatcaaagcaatatttacctgatttcatctcaggaattatttagccttctaatttcatcttctaagcactatacttatttttctttctttccttggttttggctagttttggcaaagggaaatagcaagtttgaggcaacaactacattacttgcaagaaaatcacaggtaaattgatttgacaaggtatatcttttacatgcctctttatgccacaggatcttttattgtacacactatagattgaacaatgtgatacctttatacactataaattgagtaatttgataccatttaatatattgacttgtgtgaaatttattatagggaatattttttagttaaatcttaatacgaccctcacgtgtcagtttttctttttctagtggatcgagtacataagaaatcattccatgaaggatagcgatgagactcgaattcaggattgtaaattgtcaaaactcaaaactacctcgcctgagttaactgctagggagcaaacatttatatttatttattctagttctccaagatagattgttaagtcattttttttttttgcctactttgtattcttataagtttctatgcataaaaaagttaaaaattttattggagataaagttttaagtaatatgagcacttgacaaatccaatcaacaatcaaacccattattacttttacttcatacacaaacaaatgccattttgggtaattaaccagaatttctaaaacatcatcaaaatttcttcatcatgtttccgaaaatagttatgatattttgaccaatcacatttttttatacaataatctacacctacttatccccagctgttgctggtgctttcagttgaatatgcaaggacttttaggggctcttttctagtgtttgaaacagtttaaggtttctcaaagcttcttttttttctttttttttttgagtaaggtgacaggtttctcaaagcttcctcttatcatagtctttacacttggggccatttaaggattggaaaacagaacaaaaaaggaaaaagatgctgcatactgattttgttttaaggttcatttctcacaagcattagagtaattcaggtttcttgaagcttttagtcaaatttatggtttagagcaaacactgggaaaacagaacaacacaaagatgccatgattatttaatttagaggttataagatacactcctagcatctcttgggcacaactgaagttaaaagtgcccaacagaactctaaaccgagtaaaataattttctacagctgaagttgctagcgattgattgctagtaataagaagaactataacaataaatgtagacaccaaaatttaactaaaaagccaaaaacctagtcaaactcaaatatcataaaattaagtcgatcaagaaagaacttgcaaaatgcccaaaaacaaaatttttaaaaaatctaaggattactaacgaaaaaagacatccaagacttgaatttgcaatgttttcttccatgtttttctgtgaacaactcctggaggagggaagaataactataaaaaatattaagatgaacatccccttcttttgtagcaacgtatcggagcttgtcggcatgcttaacatgcaattaaataacaataaaacgtgtatatttcaatgtctgagatctagcttagtaagctaagctgctaagggagaaatattttaactaatccctttcttttatgaaccacaggcagcttctgggtgaagaactttctggactcagcatcaaagatctaacaaatcttgaaaaccaacttgaaatgagtttgaaaggcgttcgaaagcaaaaggtaaaatccacacttttaaaattcttttgtggtcatgaaacttatgtttatatagtctacaacttatcctgaatgtattactttttcttgcaggagcagattttgactgatgaaatcagagaactaaatcagaaggttctactagaaaaaccatcagaacttcccatattctcttgtctttaaaattacccggatgattaatattgcttctgcagggcaatcttattcatcaagaaaatatagaactctataaaaaggtaaacctcattcgacaagagaatacagaattgaagaaaaaggtattaatttagagagtattgcctccacttgttgtttcaagatgtaccataaaattttgcttttacattttagtcttctaccacaagcatagatatccttgagaccttgaccttaatctaaaggagaagttacctatgccaggtttctgagcaaggatgtggaagtgaaccaaatgggggtgttcaggccacacatgcaatcagcaaaggatatgatgtacatgcaccaatcagtctccagctaagccagccacaaacacagaagagtgtaacatcaacaagcgtcatgcagcttgggtaa

SEQ ID NO: 15 - protein, 235 aa, Nicotiana tabacum
MGRGKIVIQRIDNTTSRQVTFSKRRNGLLKKAKELAILCDAEVGLIIFSSTGKLYEFSSNSMRAIIDRYNEMKEEHHNLMSPMSEVKFWQREIASLRQQLHYLQENHRQLLGEELSGLSIKDLTNLENQLEMSLKGVRKQKEQILTDEIRELNQKGNLIHQENIELYKKVNLIRQENTELKKKVSEQGCGSEPNGGVQATHAISKGYDVHAPISLQLSQPQTQKSVTSTSVMQLG

SEQ ID NO: 16 - DNA, 515 bp, synthetic construct
  misc_feature 1..515: note = synthetically-generated oligonucleotide
  source 1..515: mol_type = other DNA
aattggatccatggggagaggaaaaattgtgattcaaaggattgacaatacaacaagcaggcaagtgactttctcaaagagaagaaatgggctgctgaagaaggctaaagagctagcaattctttgtgatgctgaagttggactcattattttctccagcactggaaaactctatgaattttaataagatcttcaacacctacaccatttttttaatcactactacccattgcattgaacaaacttccaagttcttcttagcttcagattaagaaagtaccctttcttggctttgttgatgtggtaccattgtccattgtcttgtgtgtttccaaaattcatagagttttccagtgctggagaaaataatgagtccaacttcagcatcacaaagaattgctagctctttagccttcttcagcagcccatttcttctctttgagaaagtcacttgcctgcttgttgtattgtcaatcctttgaatcacaatttttcctctccccattctagaaatt

SEQ ID NO: 17 - DNA, 1194 bp, Nicotiana tabacum
  source 1..1194: mol_type = genomic DNA
atgtctcaagatggctcagcagcagcaacaatggatcggaagaaggattctcaatcaacaacaaaccctacccgaaatggtaacggtaacggtaacggcggaacagcaacaaacggcgccgtttacccgtcactcaattctctgaggttgcgtttaaaccctagtattgatcacaagtcggataattatgaagatcttcaattggattttagtccattgttatttagctcgctcgaacggtaccttccgcctactctcctcaattcgtcacgtgaccataaagttcattacatgcgggaaattctccttaagtattgtcctgaaggtgaacgtacacgtgtgcagaagcatagggaataccgacagaagattatatcaaactatcagcctctacacagggagttatataccatgcatgctgcgaatttttttgtgccatcatttcttaaggcaattagcgagaacacagaggaaagttacagaaacataatgtcggaaccctctcctggagttttaacatttgaaatgcttcaaccacgattttgtgagatgatgttggctgaggtagaaaactttgaaaggtgggtccgcgagacaaagttcagaataatgcgtcccaatacaatgaacaaatttggtgccgttcttgatgactttggcctcgaaactatgcttgacaagttgatggaagatttcattcgtcccatttcaagagttttctttactgaagttggtggatccacacttgatagccatcacggttttgttgtcgagtatgggatggatagagatgttgacttgggttttcatgttgatgactcagaagtaactttaaatgtgtgcttgggtaagcaattctctggtggagagctgttcttccgtggagtgcggtgtgagaagcatgtaaatactgaaacacaacctgaggagatctttgattactcccatgtcacggggcgtgcagtgcttcatcgtggtcgccatcggcatggtgctagagcaacaacatcggggcagaggatcaacttgttgttgtggtgcagaagttctgtctttagagagctaaggaagtaccagaaagacttttccggctggtgtgcagagtgccagcgcgagaagaaggaaaggcaacgactgtccattgctgctactaaattggagctgctgaaaagagaaggggaagctgctacatag

SEQ ID NO: 18 - DNA, 5548 bp, Nicotiana tabacum
  source 1..5548: mol_type = genomic DNA
atgtctcaagatggctcagcagcagcaacaatggatcggaagaaggattctcaatcaacaacaaaccctacccgaaatggtaacggtaacggtaacggcggaacagcaacaaacggcgccgtttacccgtcactcaattctctgaggttgcgtttaaaccctagtattgatcacaagtcggataattatgaagatcttcaattggattttagtccattgttatttagctcgctcgaacggtaccttccgcctactctcctcaattcgtcacgtgaccataaagttcattacatgcgggaaattctccttaagtattgtcctgaaggtgaacgtacacgtgtaagtatagcattttcaattttgtaaattacatttttcaattttgcgtttgtaatttgcatgtgattgttgttgttctttgtacattttgattagtgtatatattatgttagaatgtgggtagtgaattgagccattattaactgatgagaagctgaaaagggaaaacttagaagctgaactttttctgtgctttgtattgcaattttgcatgtacgttgccattaagcaatttgctgaaaaatcaactttttttatgtcgaattagttttctcaatgaaacacctttgttagtattaaaaaatagtgaaagggtggcttgtggagctctaggtacgcagagctgtgaattcagctgtaatagagtgcttatatgctgtttgatgagaatgaaaaaaacaaaagaaaataaaaagctagaaaaatctgggctttttgatggttattgctgaaaatctcgggctttgttttattctcatccattattgtcctgaacgtgaatttgcattttgcacgtggttttttttttttgtattttttaccgtggtatctaggtcagcttgtgcgtacctctattattccaccggatacctgctacctccaccggcgcaggtactaggtagctctatccactaagacttaggcggatgagaaaaaatcatctaatattattttgcctccgctatgatttgaacctgatacttcgtgattctcctcccactgaccactaggccacacccttgggtgcgacatagcttgtttctcttttcaataggcaaatgatttaataattcagggagatgtgacccgagtatagatggatataagaacaaccaaaattttggttaaaaagaacatgtgatctaagttaaacctacctggttaatttgtttataatgtggtggaatatttacaaaacatgccagtctcctaatgaagtccttgtttacccttgaactattaggaatgttgtctgctgatatagtcttctctgaatgtcctttgctttccaaacttttccttgctatttgtgagattgtactctctctctctctctcattccgaaactctatcagtacaatattgtttcatctcctctgctttaacaggtgcagaagcatagggaataccgacagaagattatatcaaactatcaggtatttacgttctgcagaagggatttgtaggttaatatttcagtgatggttgtaaatgaagacatcaatttttatactttttgttgttaagtctattaaagtaaggtaagaaagtttacgtaactttgcttgcgttttctctggagagaaatgcctttccatttaaagaattataagcatagaatgtaatgctcattgatcttcgtttagcattaatcttcttcctttctattttctaattagctttccttttattccagcctctacacagggagttatataccatgcatgctgcgaatttttttgtgccatcatttcttaaggcaattagcgagaacacagaggaaagttacagaaacataatgtcggaaccctctcctggagttttaacatttgaaatgcttcaaccacgattttgtgagatgatgttggctgaggtatcaaccggcttgctgctgttattttgcatgctaaatctagtttttctttgtagtttagcatttctgttttcttatcaggttctattgcttaggtagaaaactttgaaaggtgggtccgcgagacaaagttcagaataatgcgtcccaatacaatgaacaaatttggtgccgttcttgatgactttggcctcgaaactatgcttgacaagttgatggaagatttcattcgtcccatttcaagaggtaagaattttacttttaactgcgatgcagcaagaaaggagagaggatctgattgtgaaaattagttggctcttctcttagtcttcctctttgatatttttgtgcagttttctttactgaagttggtggatccacacttgatagccatcacggttttgttgtcgagtatgggatggatagagatgttgacttgggtaaatttttgtatgcttttgagtacgggcacacttttaccctttctttaagtgaattttttaccttatcaaaaaaaagttattgtttgtttagtacttattggggtcagctattgctttgaagtgcctcaaagcctttttggctctgtgagggacattgtcttgcatctcaaccaatttaattttcaggttttcatgttgatgactcagaagtaactttaaatgtgtgcttgggtaagcaattctctggtggagagctgttcttccgtggagtgcggtgtgagaagcatgtaaatactgaaacacaacctgaggtaattattgttccgttgtttgattaactgtgcacgagaaatctctgtttcaataggaagccatgtttccattatttcatatttatcaacaaaaaaattccattatttcctatgtatttttgtttaaatggttattgtgttgtcgaaactagtagctttttgcacagtgcccttactgtcaacaacctggtaaccaagtgtattgcaactttttcagagaaaatgctgactgtttattgtgataatttgaagtcgtgataaatgttaaacctgagtttcaaagctggatccatgacaatcttcttgatcacgtgccggaacggaaaaaggcagaggactgctcttatctctttttacttttacaagtcaaaatgattccattgatatccaccatacagtgtgaatgaacgataagcttcaggtgaaaaaacggaaacctccaactatataaagaatatgacatgtcttctttacaccagaagtacaaatgaaacaaaatgtagatttaactctccacattggactctcaaccccttcaaacatttcttctgctccttctccatattagccaattaaagaggcacaactttccattattcgcttatactggcatccaactggtggtaccttttattggcaccttactttgaatgcagatttgcttgctttatttctccatagtaatcagtgatgatgaatatatgctgtgttattaccaagtttatcagctcattctcgaagtctacatatggtttaaatactgaagaaagaatgttgttagctccacgttatttgtgtgttagagtatatatcatcaaattcaaaatacaacttatgcatgtcaagagcattcactatggatttggaataatttaacaggagatctttgattactcccatgtcacggggcgtgcagtgcttcatcgtggtcgccatcggcatggtgctagagcaacaacatcggggcagaggatcaacttgttgttgtggtgcagaaggtattagatgctgttgccttgctattttgatcgtttccactagacaatttgcttcaaacacagtcgtgaaaatttgttattgttggaatatcctgttaccgctgcattacctactgtttgaagttttaagctgtgcttcataattgtttgagaacagcagtggatgcagctctttggcaacaggttcatctgaacctagtattttttccgcggtatgtgaaaacccgctaaatttcaacaaataaaaaagggcagcccagcacacaaagcatctcgcattcacacagggtccggggaagggccgcatcctaaggggtgtgatgtagacagcctacactaattccactgctcgaaccggtgacctataggtcataccgagacaactttctcgtcgctctaaggctctccttcttaaatttcaacaaatattagatctaaacccatactttttagaagaacaatgagttcagagctaagaaccttaaaggtcaaacccatctaagtttaaatccttgtctgcctctgtttaagtatgcaatattgatgcttaactacggtctccattttatctgcatagtcctccttgatgcagatatttggatgttttccattttatttctggcatcagtgcttcaagtactgaaagtagtttatgtatatcaactttcaatgatatgaggagaagtagtgtaaagtgacggtggggaaaggagcgaatactgtcttataatcatgaaaacatgatgctgatattccttgaggcacctaaatatatgtgccatgctgagttggcaactgaaaatggcatgagttaacttggaatagcatagtttagcatgtttgtagaaacgttgattaaatggttacattatatctagatctcaactttttcccttggccattgatagtggttggtcatacagacatctactagttatcgtgatttgtcaaattttttatggatacctgattacctggtggttatggtatcactcattctcctttttcacaaaataaaagaagagttttgataaacagctgaatgcatatcatgctttctggaaaagcttttctcaagaattggagatgcacatattattgattttctttttcagtgatgataggtaacttcatataggataaattcgttttgcactaactgttttacatggacagtgtgcttcatggtattcctaaaccttgaaccatgttttgctcatgtttggcttcaaacttatcaggattaaaattgctggaggtattgctaagatgctaagaaaaatctgatttatacaaggcttcacttcatatgatgcaaaatctttgcttggagtatctaattgtaactgctttttcaagaggggatggtccggtgttggtaaaagtgcataacattatacttgttccaactaaataatttgttttacttgtcaataaaaattgtatgtaaagatatgaacttctttgctgcaactcaatcagaccagatctcaataattcggatgtcttacttttattcatcttccagaatcattgaatacctgatagttgctatctttcccttacagttctgtctttagagagctaaggaagtaccagaaagacttttccggctggtgtgcagagtgccagcgcgagaagaaggaaaggcaacgactgtccattgctgctactaaattggtaagcaataattgtctcttgtaataactttaacaaatagttgacaagctctgcttatgcattatgacattaaaaggtgaaatatagttagagctggttgaagtatttttctaaattattttgtcatttgtcctttatgttttccaggagctgctgaaaagagaaggggaagctgctacatag

SEQ ID NO: 19 - protein, 397 aa, Nicotiana tabacum
MSQDGSAAATMDRKKDSQSTTNPTRNGNGNGNGGTATNGAVYPSLNSLRLRLNPSIDHKSDNYEDLQLDFSPLLFSSLERYLPPTLLNSSRDHKVHYMREILLKYCPEGERTRVQKHREYRQKIISNYQPLHRELYTMHAANFFVPSFLKAISENTEESYRNIMSEPSPGVLTFEMLQPRFCEMMLAEVENFERWVRETKFRIMRPNTMNKFGAVLDDFGLETMLDKLMEDFIRPISRVFFTEVGGSTLDSHHGFVVEYGMDRDVDLGFHVDDSEVTLNVCLGKQFSGGELFFRGVRCEKHVNTETQPEEIFDYSHVTGRAVLHRGRHRHGARATTSGQRINLLLWCRSSVFRELRKYQKDFSGWCAECQREKKERQRLSIAATKLELLKREGEAAT

SEQ ID NO: 20 - DNA, 490 bp, synthetic construct
  misc_feature 1..490: note = synthetically-generated oligonucleotide
  source 1..490: mol_type = other DNA
aattggatccatgtctcaagatggctcagcagcagcaacaatggatcggaagaaggattctcaatcaacaacaaaccctacccgaaatggtaacggtaacggtaacggcggaacagcaacaaacggcgccgtttacccgtcactcaattctctgaggttgcgtttaaactaataagatcttcaacacctacaccatttttttaatcactactacccattgcattgaacaaacttccaagttcttcttagcttcagattaagaaagtaccctttcttggctttgttgatgtggtaccattgtccattgtcttgtgtgtttccgtttaaacgcaacctcagagaattgagtgacgggtaaacggcgccgtttgttgctgttccgccgttaccgttaccgttaccatttcgggtagggtttgttgttgattgagaatccttcttccgatccattgttgctgctgctgagccatcttgagacattctagaaatt